(19) United States
(12) Patent Application Publication
(10) Pub. No.: US 2013/0005829 A1
(43) Pub. Date: Jan. 3, 2013

(54) Segmented, epsilon-Caprolactone-Rich, Poly(epsilon-Caprolactone-co-p-Dioxanone) Copolymers for Medical Applications and Devices Therefrom

(75) Inventors: Dennis D. Jamiolkowski, Long Valley, NJ (US); Sasa Andjelic, Nanuet, NY (US); Modesto Erneta, Princeton Junction, NJ (US)

(73) Assignee: Advanced Technologies and Regenerative Medicine, LLC., Rayham, MA (US)

(21) Appl. No.: 13/249,730

(22) Filed: Oct. 14, 2011

Related U.S. Application Data
(63) Continuation-in-part of application No. 13/173,225, filed on Jun. 30, 2011.

(51) Int. Cl. A61K 31/09; A61P 31/04; C08G 63/66; A61K 47/34

(52) U.S. Cl. 514/717; 514/772.7; 525/418

(57) ABSTRACT

Novel semi-crystalline, epsilon-caprolactone-rich block copolymers of epsilon-caprolactone and p-dioxanone for long term absorbable medical applications are disclosed. The novel polymer compositions are useful for long term absorbable surgical sutures, and other medical devices. Also disclosed are compositions and methods of using tissue engineered blood vessels to repair and regenerate blood vessels of patients with vascular disease.

### FIELD OF THE INVENTION

This invention relates to novel semi-crystalline, epsilon-caprolactone-rich block copolymers of epsilon-caprolactone and p-dioxanone for long term absorbable medical applications, in particular, surgical sutures and hernia meshes. This invention also relates to tissue engineered blood vessels for treatment of vascular disease.

### BACKGROUND OF THE INVENTION

Synthetic absorbable polyesters are well known. The open and patent literature particularly describe polymers and copolymers made from glycolide, L(−)-lactide, D(+)-lactide, meso-lactide, epsilon-caprolactone, p-dioxanone, and trimethylene carbonate.

One very important application of absorbable polyesters is their use as surgical sutures. Absorbable sutures generally come in two basic forms, multifilament braids and monofilament fibers. For a polymer to function as a monofilament, it must generally possess a glass transition temperature, T_{g}, below room temperature. A low T_{g} helps to insure a low Young's modulus which in turn leads to filaments that are soft and pliable. A high T_{g} material would result in a wire-like fiber that would lead to relatively difficult handling sutures; in this art such sutures would be referred to or described as having a poor “hand”. If a polymer possesses a high T_{g}, and it is to be made into a suture, it invariably must be a construction based on multifilament yarns; a good example of this is a braid construction. It is known that monofilament sutures may have advantages versus multifilament sutures. Advantages of monofilament structures include a lower surface area, with less tissue drag during insertion into the tissue, with possibly less tissue reaction.

Other advantages include no wicking into interstices between filaments in which bacteria can move and locate. There is some thought that infectious fluids might more easily move along the length of a multifilament construction through the interstices; this of course cannot happen in monofilaments. Monofilament fiber is generally easier to manufacture as there are no braiding steps usually associated with multifilament yarns.

Absorbable monofilaments sutures have been made from poly(p-dioxanone) and other low T_{g} polymers. A very important aspect of any bioabsorbable medical device is the length of time that its mechanical properties are retained. For example, in some surgical applications it is important to retain strength for a considerable length of time to allow the body the time necessary to heal while performing its desired function. Slowly healing situations include, for example, diabetic patients or bodily areas having poor blood supply. Absorbable long term sutures have been made from conventional polymers, primarily made from lactide. Examples include a braided suture made from a high-lactide, and lactide/glycolide copolymer. In this art, those skilled in the art will appreciate that it is clear that monofilament and multifilament bioabsorbable sutures exist and that short term and long term bioabsorbable sutures exist. What does not presently exist is a bioabsorbable polymer that can be made into a suture that is soft enough to be made into a monofilament and maintain its properties post-implantation to function long term. There then remains a problem of providing such a polymer, and there is a need not only for such a polymer, but also a need for a suture made from such a polymer. It is to be understood that these polymers would also be useful in the construction of fabrics such as surgical meshes.

Besides opportunities in long term sutures and meshes, there exists opportunities for such polymers in devices that must be made from a deformable resin, ideally fabricated by known and conventional methods including as injection molding.

Crystalline block copolymers of epsilon-caprolactone and p-dioxanone are disclosed in U.S. Pat. No. 5,047,048. The copolymers covered in the patent range from about 5 to about 40 weight percent epsilon-caprolactone and the absorption profile is similar to poly(p-dioxanone). The absorbable surgical filaments have a tensile strength similar to poly(p-dioxanone) with better pliability than poly(p-dioxanone) and a lower Young's modulus of elasticity. The described copolymers are random copolymers. It is expected that fibers made from these epsilon-caprolactone/p-dioxanone copolymers, rich in p-dioxanone, will retain their mechanical properties post-implantation similar to p-dioxanone homopolymer. There then remains a need for a material that could retain mechanical properties significantly longer than that exhibited by the copolymers of '048 and that would possess Young's moduli low enough to allow fabrication into soft monofilament fibers useful as suture or mesh components. With regard to mechanical properties, U.S. Pat. No. 5,047,048 teaches away from epsilon-caprolactone/p-dioxanone block copolymers having a polymerized epsilon-caprolactone level greater than about 40 percent. They state a more preferred range between about 5 to about 30 percent, with a most preferred range being between about 5 and about 20 percent.

U.S. Pat. No. 4,791,929 and U.S. Pat. No. 4,788,979, both entitled, “Bioabsorbable Coating for a Surgical Article”, describe bioabsorbable coatings for a surgical article. The coatings comprise a copolymer manufactured from the monomer caprolactone and at least one other copolymerizable monomer. The former patent describes random copolymers while the later patent describes lower molecular weight block copolymers consistent with coating applications. The inherent viscosity of the block copolymer ranges from about 0.1 to 1.0 dl/g as measured at a concentration of 0.5 g/dl CHCl₃ at a temperature of 30° C. An aliphatic polyester of this inherent viscosity range is believed to be generally unsuitable to make strong fiber, so it appears that the inventors did not direct their invention to surgical articles in which strength is a factor.

U.S. Pat. No. 5,531,998, entitled “Polycarbonate-based Block Copolymers and Devices”, describes block copolymers based on lactones including caprolactone, but require a hard segment.

U.S. Pat. No. 5,314,989, entitled “Absorbable Composition”, describes a block copolymer for use in the fabrication of bioabsorbable articles such as monofilament surgical sutures. The copolymer is prepared by copolymerizing one or more hard phase forming monomers and 1,4-dioxan-2-one, and then polymerizing one or more hard phase forming monomers with the dioxanone-containing copolymer. The materials of this invention require a hard phase.

Similarly, U.S. Pat. No. 5,522,841, entitled “Absorbable Block Copolymers and Surgical Articles Fabricated Therefrom”, describes absorbable surgical articles formed from a block copolymer having one of the blocks made from hard phase forming monomers and another of the blocks made from random copolymers of soft phase forming monomers. Hard phase forming monomers are said to include glycolide and lactide while soft phase forming monomers include 1,4-dioxane-2-one and 1,3-dioxane-2-one and caprolactone.

U.S. Pat. No. 5,705,181, entitled “Method of Making Absorbable Polymer Blends of Polylactides, Polycaprolactone and Polydioxanone”, describes absorbable binary and tertiary blends of homopolymers and copolymers of poly(lactide), poly(glycolide), poly(ε-caprolactone), and poly(p-dioxanone). These materials are blends and not copolymers.

U.S. Pat. No. 5,133,739 describes block copolymers prepared from caprolactone and glycolide having a hard phase. US 2009/0264040A1 describes melt blown nonwoven materials prepared from caprolactone/glycolide copolymers. Although both of these are directed towards absorbable materials containing polymerized caprolactone, they absorb rather quickly and thus are not useful for long term implants.

Another area of concern is cardiovascular-related disorders. Cardiovascular-related disorders are a leading cause of death in developed countries. In the US alone, one cardiovascular death occurs every 34 seconds and cardiovascular disease-related costs are approximately $250 billion. Current methods for treatment of vascular disease include chemotherapeutic regimens, angioplasty, insertion of stents, reconstructive surgery, bypass grafts, resection of affected tissues, or amputation. Unfortunately, for many patients, such interventions show only limited success, and many patients experience a worsening of the conditions or symptoms.

These diseases often require reconstruction and replacement of blood vessels. Currently, the most popular source of replacement vessels is autologous arteries and veins. Such autologous vessels, however, are in short supply or are not suitable especially in patients who have had vessel disease or previous surgeries.

Synthetic grafts made of materials such as polytetrafluoroethylene (PTFE) and Dacron are popular vascular substitutes. Despite their popularity, synthetic materials are not suitable for small diameter grafts or in areas of low blood flow. Material-related problems such as stenosis, thromboembolization, calcium deposition, and infection have also been demonstrated.

Therefore, there is a clinical need for biocompatible and biodegradable structural matrices that facilitate tissue infiltration to repair/regenerate diseased or damaged tissue. In general, the clinical approaches to repair damaged or diseased blood vessel tissue do not substantially restore their original function. Thus, there remains a strong need for alternative approaches for tissue repair/regeneration that avoid the common problems associated with current clinical approaches.

The emergence of tissue engineering may offer alternative approaches to repair and regenerate damaged/diseased tissue. Tissue engineering strategies have explored the use of biomaterials in combination with cells, growth factors, bioactives, and bioreactor processes to develop biological substitutes that ultimately can restore or improve tissue function. The use of colonizable and remodelable scaffolding materials has been studied extensively as tissue templates, conduits, barriers, and reservoirs. In particular, synthetic and natural materials in the form of foams and textiles have been used in vitro and in vivo to reconstruct/regenerate biological tissue, as well as deliver agents for inducing tissue growth.

Such tissue-engineered blood vessels (TEBVs) have been successfully fabricated in vitro and have been used in animal models. However, there has been very limited clinical success.

Regardless of the composition of the scaffold and the targeted tissue, the template must possess some fundamental characteristics. The scaffold must be biocompatible, possess sufficient mechanical properties to resist the physical forces applied at the time of surgery, porous enough to allow cell invasion, or growth, easily sterilized, able to be remodeled by invading tissue, and degradable as the new tissue is being formed. Furthermore, the scaffold may be fixed to the surrounding tissue via mechanical means, fixation devices, or adhesives. So far, conventional materials, alone or in combination, lack one or more of the above criteria. Accordingly, there is a need for scaffolds that can resolve the potential pitfalls of conventional materials.

There is a need in this art for novel, long term bioabsorbable sutures that have good handling characteristics and strength retention. There is a further need in this art for novel bioabsorbable polymer compositions for manufacturing such sutures and other bioabsorbable medical devices.

### SUMMARY OF THE INVENTION

Novel semi-crystalline, epsilon-caprolactone-rich block copolymers of epsilon-caprolactone and p-dioxanone for long term absorbable medical applications are disclosed. The novel segmented, semicrystalline, synthetic, absorbable copolymers of the present invention consist of lactone monomers selected from the group consisting of p-dioxanone and epsilon-caprolactone, wherein the epsilon-caprolactone is a major component.

Another aspect of the present invention is a long term bioabsorbable suture made from the above-described copolymer.

Yet another aspect of the present invention is a bioabsorbable medical device made from the above described suture.

Still yet another aspect of the present invention is a method of manufacturing a medical device from said novel copolymers.

A further aspect of the present invention is a method of performing a surgical procedure wherein a medical device made from the novel copolymers of the present invention is implanted in tissue in a patient.

The invention also relates to a tissue engineered blood vessel (TEBV) comprising a scaffold having an inner braided mesh tube having an inner surface and an outer surface, a melt blown sheet on the outer surface of the inner braided mesh tube, and an outer braided mesh tube on the melt blown sheet. Furthermore, the scaffold of the TEBV may be combined with one or more of cells, cell sheets, cell lysate, minced tissue, and cultured with or without a bioreactor process. Such tissue engineered blood vessels may be used to repair or replace a native blood vessel that has been damaged or diseased.

These and other aspects and advantages of the present invention will become more apparent from the following description and accompanying drawings.

### BRIEF DESCRIPTION OF THE DRAWINGS

FIG. 1a Histology of Hematoxylin/Eosin (H&E) stained images after 7 days of culturing Rat smooth muscle cells (SMC) on poly(p-dioxanone) (PDS) melt blown scaffolds.

FIG. 1b Histology of Hematoxylin/Eosin (H&E) stained images after 7 days of culturing Rat smooth muscle cells (SMC) on 75/25 poly(glycolide-co-caprolactone) (PGA/PCL) melt blown scaffolds.

FIG. 2 DNA contents of Human Umbilical Tissue cells (hUTC) on collagen coated PDO melt blown scaffolds and PDO melt blown scaffolds.

FIG. 3 DNA contents in three scaffolds (p-dioxanone) (PDO) melt blown scaffold, 90/10 PGA/PLA needle punched scaffold, 65/35 PGA/PCL foam) that were evaluated for supporting human internal mammary arterial (iMA) cells (iMAC).

FIG. 4a H&E stained image of iMA cells seeded on a 65/35 PGA/PCL foam at 1 day.

FIG. 4b H&E stained image of iMA cells seeded on a 65/35 PGA/PCL foam at 7 days.

FIG. 4c H&E stained image of iMA cells seeded on a 90/10 PGA/PLA needle punched scaffold at 1 day.

FIG. 4d H&E stained image of iMA cells seeded on a 90/10 PGA/PLA needle punched scaffold at 7 days.

FIG. 4e H&E stained image of iMA cells seeded on a PDO melt blown scaffold at 1 day.

FIG. 4f H&E stained image of iMA cells seeded on a PDO melt blown scaffold at 7 days.

FIG. 5 Procedures for generating a braided mesh/rolled melt blown 9/91 Cap/PDO/Braided mesh scaffold.

FIG. 6 SEM of a braided mesh/rolled melt blown 9/91 Cap/PDO/Braided mesh scaffold.

FIG. 7 Cross-sectional SEM view of a braided mesh/rolled melt blown 9/9 Cap/PDO/Braided mesh scaffold.

FIG. 8a H&E stained image of a scaffold of a braided mesh/a rolled melt blown (PDO/PCL)/a braided mesh with hUTC cultured in bioreactor cassette for 7 days.

FIG. 8b H&E stained image of a scaffold of a braided mesh/a rolled melt blown (PDO/PCL)/a braided mesh with hUTC cultured in bioreactor cassette for 7 days.

FIG. 8c H&E stained image of a scaffold of a braided mesh/a rolled melt blown (PDO/PCL)/a braided mesh with hUTC cultured in bioreactor cassette for 7 days.

FIG. 8d H&E stained image of a scaffold of a braided mesh/a rolled melt blown (PDO/PCL)/a braided mesh with hUTC cultured in bioreactor cassette for 7 days.

### DETAILED DESCRIPTION OF INVENTION

Poly(epsilon-caprolactone) is a low Tg (−60° C.) semi-crystalline polyester. Although this material has a low elastic modulus it does not absorb quickly enough for many key surgical applications, i.e., it lasts too long in vivo. It has been found, however, that certain epsilon-caprolactone-rich copolymers are particularly useful for the present application. For instance, a 91/9 mol/mol poly(epsilon-caprolactone-co-p-dioxanone) copolymer [91/9 Cap/PDO] was prepared in a sequential addition type of polymerization starting with a first stage charge of epsilon-caprolactone followed by a subsequent second stage of p-dioxanone. The total initial charge was 75/25 mol/mol epsilon-caprolactone/p-dioxanone. Due to incomplete conversion of monomer-to-polymer and difference in reactivity, it is not uncommon to have the final (coolymer composition differ from the feed composition. The final composition of the copolymer was found to be 91/9 mol/mol epsilon-caprolactone/p-dioxanone. See EXAMPLE 3 for the details of this copolymerization.

The present invention is directed towards copolymers of epsilon-caprolactone and p-dioxanone. More specifically, this class of copolymers rich in epsilon-caprolactone and made to have a blocky sequence distribution, that is non-random. In epsilon-caprolactone/p-dioxanone copolymers in which the majority of the material is based on p-dioxanone, there is present a breakdown rate which is too fast to be useful in long term applications. The compositions must be rich in epsilon-caprolactone, e.g., having a polymerized epsilon-caprolactone content of 50 percent or greater.

Dimensional stability in a fiber used to manufacture a surgical suture is very important to prevent shrinkage, both in the sterile package before use, as well as in the patient after surgical implantation. Dimensional stability in low T_{g} material can be achieved by crystallization of the formed article. Regarding the phenomena of crystallization of copolymers, a number of factors play important roles. These factors include overall chemical composition and the sequence distribution.

Although the overall level of crystallinity (and the T_{g} of the material) plays a role in dimensional stability, it is important to realize that the rate of crystallization is critical to processing. If a low Tg material is processed and it rate of crystallization is very slow, it is very difficult to maintain dimensional tolerances since shrinkage and warpage easily occur. Fast crystallization is thus an advantage. To increase the rate of crystallization of a copolymer of given overall chemical composition, a block structure would be preferable over a random sequence distribution. However, achieving this with the two lactone monomers, epsilon-caprolactone and p-dioxanone is known to be very difficult.

Poly(p-dioxanone) has a low ceiling temperature, accordingly at elevated temperatures it tends to exist with a high fraction of monomer at equilibrium. When starting with fully polymerized material at elevated temperatures, it “depolymerizes” thereby resulting in a combination of polymer and regenerated monomer. Regenerated equilibrium monomer levels for poly(p-dioxanone) can be rather high, approaching 30 to 50 percent at reaction temperatures of 110 to 160° C.

On the other hand, it is quite difficult to polymerize epsilon-caprolactone at temperatures lower than about 160° C. There then exists a problem as to how to achieve polymerization of these two co-monomers to produce a block structure with high enough molecular weight so as to result in products having good mechanical properties.

The novel copolymers of the present invention are prepared by first polymerizing the epsilon-caprolactone monomer at temperatures between about 170° C. and about 240° C. Temperatures between about 185 and about 195° C. are particularly advantageous. Although a monofunctional alcohol such as dodecanol might be used for initiation, a diol such as diethylene glycol has been found to work well. Combinations of mono-functional and di-functional, or multifunctional conventional initiators may also be used. Reaction times can vary with catalyst level. Suitable catalysts include conventional catalysts such as stannous octoate. The catalyst may be used at a monomer/catalyst level ranging from about 10,000/1 to about 300,000/1, with a preferred level of about 25,000/1 to about 100,000/1. After the completion of this first stage of the polymerization, the temperature is lowered substantially, but still above a temperature of 60° C. Once the temperature is lowered, for example to 150° C., p-dioxanone monomer can be added to the reactor; this can be conveniently done by pre-melting this second monomer and adding it in a molten form. Once the p-dioxanone monomer is added, the temperature is brought to about 110° C. to complete the co-polymerization.

Alternately, once the p-dioxanone monomer is added, the temperature can be brought to about 110° C., maintained at this temperature for some period of time (e.g. 3 to 4 hours), followed by polymer discharge into suitable containers for subsequent low temperature polymerization (e.g., 80° C.) for an extended period of time to complete the co-polymerization. Higher monomer-to-polymer conversions may be possible utilizing this alternate low temperature finishing approach.

It will be clear to one skilled in the art that various alternate polymerization approaches are possible and still produce the copolymer of the subject invention. One might then consider a process in which the reaction temperature after the initial stage of polymerizing the epsilon-caprolactone is dropped immediately to 110° C. prior to the addition of the p-dioxanone monomer. Again, one skilled in the art can provide a variety of alternate polymerization schemes.

Poly(epsilon-caprolactone-co-p-dioxanone) copolymers rich in polymerized epsilon-caprolactone having levels of incorporated p-dioxanone greater than about 40 mole percent are unsuitable for copolymers of the present invention because of crystallization difficulties. Poly(epsilon-caprolactone-co-p-dioxanone) copolymers comprising a polymerized epsilon-caprolactone having a molar level between 60 to 95 percent and a polymerized p-dioxanone molar level between 5 to 40 percent are useful in the practice of the present invention. This class of copolymers, the poly(epsilon-caprolactone-co-p-dioxanone) family rich epsilon-caprolactone, should ideally contain about 10 to about 30 mole percent of polymerized p-dioxanone.

The copolymers of the subject invention are semicrystalline in nature, having a crystallinity level ranging from about 10 to about 50 percent. They will have a molecular weight sufficiently high to allow the medical devices formed therefrom to effectively have the mechanical properties needed to perform their intended function. For melt blown nonwoven structure the molecular weight may be a little lower, and for extruded fibers, they may be a little higher. Typically, for example, the molecular weight of the copolymers of the subject invention will be such so as to exhibit inherent viscosities as measured in hexafluoroisopropanol (HFIP, or hexafluoro-2-propanol) at 25° C. and at a concentration of 0.1 g/dL between about 0.5 to about 2.5 dL/g. The surgical suture made from the novel copolymers of the present invention preferably is a monofilament with a Young's modulus of less than about 150,000 psi. In one embodiment, the copolymer has a glass transition temperature below about 25′C. The novel copolymers of the present invention will preferably have an absorption time between about 6 and about 24 months.

In one embodiment, the medical devices made of the copolymers of the present invention may contain conventional active ingredients, such as antimicrobials, antibiotics, therapeutic agents, hemostatic agents, radio-opaque materials, tissue growth factors, and combinations thereof. In one embodiment the antimicrobial is Triclosan, PHMB, silver and silver derivatives or any other bio-active agent.

The copolymers of the subject invention can be melt extruded by a variety of conventional means. Monofilament fiber formation can be accomplished by melt extrusion followed by extrudate drawing with or without annealing. Multifilament fiber formation is possible by conventional means. Methods of manufacturing monofilament and multifilament braided sutures are disclosed in U.S. Pat. No. 5,133,739, entitled “Segmented Copolymers of epsilon-Caprolactone and Glycolide” and U.S. Pat. No. 6,712,838 entitled “Braided Suture with Improved Knot Strength and Process to Produce Same”, are incorporated by reference herein in their entirety.

The copolymers of the present invention may be used to manufacture conventional medical devices in addition to sutures using conventional processes. For example, injection molding might be accomplished after allowing the copolymer to crystallize in the mold; alternately biocompatible nucleating agents might be added to the copolymer to reduce cycle time. The medical devices may include, in addition to meshes, the following conventional devices meshes, tissue repair fabrics, suture anchors, stents, orthopedic implants, staples, tacks, fasteners, suture clips, etc.

Sutures made from the copolymers of the present invention may be used in conventional surgical procedures to approximate tissue or affix tissue to medical devices. Typically, after a patient is prepared for surgery in a conventional matter, including swabbing the outer skin with antimicrobial solutions and anesthetizing the patient, the surgeon will make the required incisions, and, after performing the required procedure proceed to approximate tissue using the long-term absorbable sutures of the present invention (in particular monofilament sutures) made from the novel copolymers of the present invention. In addition to tissue approximation, the sutures may be used to affix implanted medical devices to tissue in a conventional manner. After the incisions are approximated, and the procedure is completed, the patient is then moved to a recovery area. The long-term absorbable sutures of the present invention in the patient retain their strength in vivo for the required time to allow effective healing and recovery.

Also disclosed herein as an invention is a tissue engineered blood vessel (TEBV) comprised of an inner braided mesh tube having an inner surface and an outer surface, a melt blown sheet disposed on the outer surface of the inner braided mesh tube, and an outer braided mesh tube disposed on the melt blown sheet. Furthermore, the TEBV may be combined with one or more of cells, cell sheets, cell lysate, minced tissue, and cultured with or without a bioreactor process. Such tissue engineered blood vessels may be used to repair or replace a native blood vessel that has been damaged or diseased. In tissue engineering, the rate of resorption of the scaffold by the body preferably approximates the rate of replacement of the scaffold by tissue. That is to say, the rate of resorption of the scaffold relative to the rate of replacement of the scaffold by tissue must be such that the structural integrity, e.g. strength, required of the scaffold is maintained for the required period of time. If the scaffold degrades and is absorbed unacceptably faster than the scaffold is replaced by tissue growing therein, the scaffold may exhibit a loss of strength and failure of the device may occur. Additional surgery then may be required to remove the failed scaffold and to repair damaged tissue. The TEBV described herein advantageously balances the properties of biodegradability, resorption, structural integrity over time, and the ability to facilitate tissue in-growth, each of which is desirable, useful, or necessary in tissue regeneration or repair.

The braided mesh tubes and the melt blown sheet are prepared from biocompatible, biodegradable polymers. The biodegradable polymers readily break down into small segments when exposed to moist body tissue. The segments then are either absorbed by or passed from the body. More particularly, the biodegraded segments do not elicit permanent chronic foreign body reaction, because they are absorbed by the body or passed from the body such that no permanent trace or residual of the segment is retained by the body. For the purposes of this invention the terms bioabsorbable and biodegradable are used interchangeably.

The biocompatible, biodegradable polymers may be natural, modified natural, or synthetic biodegradable polymers, including homopolymers, copolymers, and block polymers, linear or branched, segmented or random, as well as combinations thereof. Particularly well suited synthetic biodegradable polymers are aliphatic polyesters which include but are not limited to homopolymers and copolymers of lactide (which includes D(−)-lactic acid, L(+)-lactic acid, L(−)-lactide, D(+)-lactide, and meso-lactide), glycolide (including glycolic acid), epsilon-caprolactone, p-dioxanone (1,4-dioxan-2-one), and trimethylene carbonate (1,3-dioxan-2-one).

For a tubular structure to fulfill the requirements set out for a successful TEBV (or similar tubular device or sheet stock scaffold), it must possess certain key properties. The structure as a whole must exhibit an ability to allow radial expansion in a pulsatile manner similar to what is seen in human arteries. This means, in part, to match the elastic modulus of arteries. An elastic modulus of 1 to 5 MPa would be appropriate, and an elastic modulus lower than that exhibited by polyp-dioxanone) is sought.

Moreover, the retention time of mechanical properties, post-implantation, must be sufficient for the intended use. If the device is to be pre-seeded with cells and the cells allowed to propagate prior to implantation of the device, then the pre-seeded device must withstand the rigors of surgical implantation, including fixation at both ends. If the device is to be implanted without being pre-seeded with cells, the device must possess sufficient retention of mechanical properties to allow appropriate cellular in-growth to be functional. In general, a retention time of mechanical properties greater than that exhibited by poly(p-dioxanone) is sought. It is to be understood that a successful material must still absorb in an appropriate time frame, i.e. 6 to 18 months, and typically not more than about 24 months. One material that may come under the consideration of some researchers is poly(epsilon-caprolactone). This material, although having a low elastic modulus, does not absorb quickly enough to meet requirements.

Dimensional stability of a low modulus polymeric fiber that is not cross-linked as in rubber fibers is generally achieved by inducing some measure of crystallinity. It is to be understood that the rate at which a polymer crystallizes is also very important during the process of melt blowing the nonwoven fabric itself. If it crystallizes too slowly, the low modulus nature of the material cannot support the structure and the fabric collapses onto itself resulting into a film-like structure. In one embodiment, a polymer has a glass transition temperature below 25° C.

In some instances, it may be desirable to have the fibers making up the nonwoven fabric quite small in diameter; i.e. 2 to 6 microns in diameter or lower. To achieve this, it may be necessary to limit the molecular weight of the resin. In one embodiment, a polymer exhibits an inherent viscosity between 0.5 and 2.0 dL/g.

Existing materials are deficient in meeting the new challenges presented. Two copolymer systems that meet the challenging requirements set forth above have unexpectedly been discovered. These systems are both based on the lactone monomers p-dioxanone and epsilon-caprolactone. In one case, the monomer ratio favors p-dioxanone; that is, p-dioxanone-rich poly(epsilon-caprolactone-co-p-dioxanone). In the other case, the monomer ratio favors epsilon-caprolactone; that is, epsilon-caprolactone-rich poly(epsilon-caprolactone-co-p-dioxanone).

### Copolymer I

### Segmented, p-dioxanone-Rich, Poly(epsilon-caprolactone-co-p-dioxanone) Copolymers [PDO-Rich Cap/PDO]

Poly(p-dioxanone) is a low Tg (−11° C.) semi-crystalline polyester finding extensive utility as a suture material and as injection molded implantable medical devices. It will be understood by one having ordinary skill in the art that the level of crystallinity needed to achieve dimensional stability in the resulting fabric will depend on the glass transition temperature of the (coolymer. That is, to avoid fabric shrinkage, warpage, buckling, and other consequences of dimensional instability, it is important to provide some level of crystallinity to counteract the phenomena. The level of crystallinity that is needed for a particular material of given glass transition temperature with given molecular orientation can be experimentally determined by one having ordinary skill in the art. The level for crystallinity required to achieve dimensional stability in melt blown nonwoven fabrics may be a minimum of about 20 percent in polymeric materials possessing glass transition temperatures of about minus 20° C.

Besides the level of crystallinity, the rate of crystallization is very important in the melt blown nonwoven process. If a material crystallizes too slowly, especially if it possesses a glass transition temperature below room temperature, the resulting nonwoven product may have a collapsed architecture, closer to a film than a fabric. A slow-to-crystallize (coolymer will be quite difficult to process into desired structures.

It would be advantageous to have a material exhibiting a greater reversible extensibility (i.e. elasticity) and a lower modulus than poly(p-dioxanone). Certain p-dioxanone-rich copolymers are particularly useful for this application. Specifically, a 9/91 mol/mol poly(epsilon-caprolactone-co-p-dioxanone) copolymer [9/91 Cap/PDO] was prepared in a sequential addition type of polymerization starting with a first stage charge of epsilon-caprolactone followed by a subsequent second stage of p-dioxanone. The total initial charge was 7.5/92.5 mol/mol epsilon-caprolactone/p-dioxanone. See EXAMPLE 2 for the details of this copolymerization.

Poly(epsilon-caprolactone-co-p-dioxanone) copolymers rich in polymerized p-dioxanone having levels of incorporated epsilon-caprolactone greater than about 15 mole percent are unsuitable for the present application, because it is difficult to prepare melt blown nonwoven fabrics from such copolymers. It is speculated that this may be because p-dioxanone-rich poly(epsilon-caprolactone-co-p-dioxanone) copolymers having greater than about 15 mole percent of incorporated epsilon-caprolactone exhibit too high an elastic modulus resulting in “snap-back” of extruded fibers leading to very lumpy unsuitable fabric. See EXAMPLES 1 and 5 for the synthesis and processing details, respectively.

### Copolymer II

### Segmented, epsilon-caprolactone-Rich, Poly(epsilon-caprolactone-co-p-dioxanone) Copolymers [Cap-Rich Cap/PDO]

Poly(epsilon-caprolactone) is also a low Tg (−60° C.) semi-crystalline polyester. As previously discussed, this material, although having a low elastic modulus, does not absorb quickly enough to meet requirements. It has been found, however, that certain epsilon-caprolactone-rich copolymers are particularly useful for the present application. Specifically, a 91/9 mol/mol poly(epsilon-caprolactone-co-p-dioxanone) copolymer [91/9 Cap/PDO] was prepared in a sequential addition type of polymerization starting with a first stage charge of epsilon-caprolactone followed by a subsequent second stage of p-dioxanone. The total initial charge was 75/25 mol/mol epsilon-caprolactone/p-dioxanone. Due to incomplete conversion of monomer-to-polymer and difference in reactivity, it is not uncommon to have the final (coolymer composition differ from the feed composition. The final composition of the copolymer was found to be 91/9 mol/mol epsilon-caprolactone/p-dioxanone. See EXAMPLE 3 for the details of this copolymerization.

Poly(epsilon-caprolactone-co-p-dioxanone) copolymers rich in polymerized epsilon-caprolactone having levels of incorporated p-dioxanone greater than about 20 mole percent are unsuitable for the present application, because it is difficult to prepare melt blown nonwoven fabrics from such copolymers. It is speculated that this may be because epsilon-caprolactone-rich poly(epsilon-caprolactone-co-p-dioxanone) copolymers having levels of incorporated p-dioxanone greater than about 20 mole percent do not crystallize quickly enough leading to unsuitable fabric.

As discussed herein, suitable synthetic bioabsorbable polymers for the present invention include poly(p-dioxanone) homopolymer (PDO) and p-dioxanone/epsilon-caprolactone segmented copolymers rich in p-dioxanone. The latter class of polymers, the poly(p-dioxanone-co-epsilon-caprolactone) family rich in p-dioxanone should ideally contain up to about 15 mole percent of polymerized epsilon-caprolactone.

Additionally, p-dioxanone/epsilon-caprolactone segmented copolymers rich in epsilon-caprolactone are useful in practicing the present invention. This class of polymers, the poly(p-dioxanone-co-epsilon-caprolactone) family rich epsilon-caprolactone, should ideally contain up to about 20 mole percent of polymerized p-dioxanone.

Other polymer systems that may be advantageously employed include the poly(lactide-co-epsilon-caprolactone) family of materials. Within this class, the copolymers rich in polymerized lactide having about 99 to about 65 mole percent polymerized lactide and the copolymers rich in polymerized epsilon-caprolactone having about 99 to about 85 mole percent polymerized epsilon-caprolactone are useful.

Other polymer systems that may be employed include the poly(lactide-co-p-dioxanone) family of materials. Within this class, the copolymers rich in polymerized lactide having about 99 to about 85 mole percent polymerized lactide and the copolymers rich in polymerized p-dioxanone having about 99 to about 80 mole percent polymerized p-dioxanone are useful. It is to be understood that the copolymers in this poly(lactide-co-p-dioxanone) family of materials rich in polymerized lactide maybe more useful where a stiffer material is required.

Other polymer systems that may be employed include the poly(lactide-co-glycolide) family of materials. Within this class, the copolymers rich in polymerized lactide having about 99 to about 85 mole percent polymerized lactide and the copolymers rich in polymerized glycolide having about 99 to about 80 mole percent polymerized glycolide are useful. It is to be understood that the copolymers in this poly(lactide-co-glycolide) family of materials rich in polymerized lactide maybe more useful where a stiffer material is required. Likewise, the copolymers in this poly(lactide-co-glycolide) family of materials rich in polymerized glycolide maybe more useful when a faster absorption time is required.

Another polymer class that may be employed includes the poly(glycolide-co-epsilon-caprolactone) family of materials. Within this class, the copolymers rich in polymerized glycolide having about 99 to about 70 mole percent polymerized glycolide and the copolymers rich in polymerized epsilon-caprolactone having about 99 to about 85 mole percent polymerized epsilon-caprolactone are useful. It is to be understood that the copolymers in this poly(glycolide-co-epsilon-caprolactone) family of materials rich in polymerized glycolide maybe more useful when a faster absorption time is required. Likewise, the copolymers in this poly(glycolide-co-epsilon-caprolactone) family of materials, rich in polymerized epsilon-caprolactone, maybe more useful when a softer material is required.

Suitable natural polymers include, but are not limited to collagen, atelocollagen, elastic, and fibrin and combinations thereof. In one embodiment, the natural polymer is collagen. In yet another embodiment, the combination of natural polymer is an acellular omental matrix.

In accordance herewith, a melt blown nonwoven process having utility herein will now be described. A typical system for use in a melt blown nonwoven process consists of the following elements: an extruder, a transfer line, a die assembly, hot air generator, a web formation system, and a winding system.

As is well known to those skilled in the art, an extruder consists of a heated barrel with a rotating screw positioned within the barrel. The main function of the extruder is to melt the copolymer pellets or granules and feed them to the next element. The forward movement of the pellets in the extruder is along the hot walls of the barrel between the flights of the screw. The melting of the pellets in the extruder results from the heat and friction of the viscous flow and the mechanical action between the screw and the walls of the barrel. The transfer line will move molten polymer toward the die assembly. The transfer line may include a metering pump in some designs. The metering pump may be a positive-displacement, constant-volume device for uniform melt delivery to the die assembly.

The die assembly is a critical element of the melt blown process. It has three distinct components: a copolymer feed distribution system, spinnerets (capillary holes), and an air distribution system. The copolymer feed distribution introduces the molten copolymer from the transfer line to distribution channels/plates to feed each individual capillary hole uniformly and is thermal controlled. From the feed distribution channel the copolymer melt goes directly to the die capillary. The copolymer melt is extruded from these holes to form filament strands which are subsequently attenuated by hot air to form fine fibers. During processing, the entire die assembly is heated section-wise using external heaters to attain the desired processing temperatures. In one embodiment, a die temperature of about 210 to 280° C. for CAP/GLY 25/75 copolymer, about 110 to 210° C. for PDO/CAP 92.5/7.5 copolymer, and 120 to 220° C. for PDS homopolymer is useful. In another embodiment, a die temperature range is from about 210° C. to about 260° C. for CAP/GLY 25/75 copolymer, about 150° C. to about 200° C. for PDO/CAP 92.5/7.5 copolymer, and about 160° C. to about 210° C. for PDS homopolymer. In another embodiment, a die pressure of about 100 to 2,000 psi is useful. In another embodiment, a die pressure range is from about 100 to about 1200 psi.

The air distribution system supplies the high velocity hot air. The high velocity air is generated using an air compressor. The compressed air is passed through a heat exchange unit, such as an electrical or gas heated furnace, to heat the air to desired processing temperatures. In one embodiment, an air temperature of about 200° C. to 350° C. for CAP/GLY 25/75 copolymer, about 180 to 300° C. for PDO/CAP 92.5/7.5 copolymer, and about 180 to 300° C. for PDS homopolymer is useful. In another embodiment, an air temperatures range is from about 220° C. to about 300° C. for CAP/GLY 25/75 copolymer, about 200° C. to about 270° C. for PDO/CAP 92.5/7.5 copolymer, and about 200 to about 270° C. for PDS homopolymer. In another embodiment, an air pressure of about 5 to 50 psi is useful, and in another embodiment an air pressure range is from about 5 to about 30 psi. It should be recognized that the air temperature and the air pressure may be somewhat equipment dependent, but can be determined through appropriate experiment.

As soon as the molten copolymer is extruded from the die holes, high velocity hot air streams attenuate the copolymer streams to form microfibers. With the equipment employed, a screw speed of about 1 to 100 RPM is adequate. As the hot air stream containing the microfibers progresses toward the collector screen, it draws in a large amount of surrounding air that cools and solidifies the fibers. The solidified fibers subsequently get laid randomly onto the collecting screen, forming a self-bonded web. The collector speed and the collector distance from the die nosepiece can be varied to produce a variety of melt blown webs. With the equipment employed, a collector speed of about 0.1 to 100 m/min is adequate. Typically, a vacuum is applied to the inside of the collector screen to withdraw the hot air and enhance the fiber laying process.

The melt blown web is typically wound onto a tubular core and may be processed further according to the end-use requirement. In one embodiment, the nonwoven construct formed by the melt blown extrusion of the aforementioned copolymer is comprised of microfibers having a fiber diameter ranging from about 1 to 8 micrometres. In another embodiment, the microfibers have a fiber diameter ranging from about 1 to 6 micrometres.

The melt blown process used to synthesize the TEBVs of the present invention is advantageous with respect to other processes, including electrostatic spinning, for various reasons. For example, the melt blown process may be better for the environment than other processes because it does not need a solvent to dissolve a polymer. Another advantage is that the melt blown process is a one-step process wherein the molten polymer resin is blown by high speed air onto a collector such as a conveyor belt or a take-up machine to form a nonwoven fabric. Moreover, the diameters of melt blown fibers are in the range of 0.1 micron to 50 microns. A combination of the broad range fibers provides a scaffold having large pores and porosity. Furthermore, composite scaffolds having micro/nano scale fibers can be produced using a combination of a melt blown and an electrospun scaffold. The electrospun scaffold may be used as a barrier, as it possesses much smaller pore sizes which can impede transport from one side to the other. Another advantage is that the rolling process does not require glue for the graft to keep its tubular shape, and the rolling process does not need sutures to reinforce the strength of the graft.

The TEBV has overall dimensions that reflect desired ranges that, in combination with the one or more of cells, cell sheets, cell lysate, minced tissue, and a bioreactor process, will replace a small diameter, damaged or diseased vein or artery blood vessel. Desirable dimensions include but are not limited to: internal diameter (3-7 mm preferable, 4-6 mm most preferable); wall thickness (0.1-1 mm preferable, 0.2-0.7 mm most preferable); and length (1-20 cm preferable, 2-10 cm most preferable). The table below shows how the properties of a Poly(p-dioxanone) construct align with those of a natural vessel.

| | Internal | Wall | | | Burst | Suture | Tensile |
|---|---|---|---|---|---|---|---|
| | Diameter | Thickness | Length | Compliance | Pressure | retention | (peak |
| | (mm) | (mm) | (cm) | (%) | (mmHg) | (gmf) | stress) |
| PDO | 2 & 5 | 0.5 | 1-20 | 0.5-1 | 1500-2500 | 310 | 5 MPa |
| Vessel | 2 & 5 | 0.5-0.7 | 1-20 | 0.2-10 | 1500-4500 | 100-500 | 2-20 MPa |

The TEBV has physical properties that reflect desired ranges that, in conjunction with one or more of cells, cell sheets, cell lysate, minced tissue, and a bioreactor process, will replace a small diameter, damaged or diseased vein or artery blood vessel. Desirable physical properties include but are not limited to: compliance (0.2-10 percent preferable, 0.7-7 percent most preferable); suture retention strength (100 gm-4 Kg preferable, 100-300 gm most preferable); burst strength/pressure (1000-4500 mm Hg preferable, 1500-4500 mm Hg most preferable with greater than 100 mm Hg during the bioreactor process); kink resistance (resist kinking during handling during all stages of process, including cell seeding, bioreactor, implantation, life of patient); and in-vitro strength retention (1 day-1 yr maintain enough strength until cell and extracellular matrix (“ECM”) growth overcomes physical property losses of TEBV; 1 day-3 mos under bioreactor “flow” conditions preferable). The TEBV should also have desirable tensile properties (radial and axial) that include but are not limited to: elastic modulus (MPa) of longitudinal/axial (1-200 preferable; 5-100 most preferable) and orthogonal/radial (0.1-100 preferable, 0.5-50 most preferable) and random (0.1-100 preferable, 0.5-50 most preferable) and wet/longitudinal (5-100 preferable, 25-75 preferable); a peak stress (MPa) of longitudinal/axial (1-30 preferable; 2-20 most preferable) and orthogonal/radial (0.5-15 n preferable, 1-10 most preferable) and random (0.5-15 preferable, 1-10 most preferable) and wet/long (1-30 preferable; 2-20 most preferable); failure strain (%) of longitudinal/axial (1-200 preferable; 5-75 most preferable) and orthogonal/radial (5-400 preferable, 10-300 most preferable) and random (5-400 preferable, 10-300 most preferable) and wet/long (1-200 preferable; 20-100 most preferable).

The TEBV has morphology that reflects desired ranges that, in conjunction with one or more of cells, cell sheets, cell lysate, minced tissue, and a bioreactor process, will replace a small diameter, damaged or diseased vein or artery blood vessel. Desirable morphology includes but is not limited to: pore size (1-200 um preferable, most preferable less than 100 um); porosity (40-98 percent preferable, most preferable 60-95 percent); surface area/vol (0.1-7 m²/cm³ preferable, most preferable 0.3-5.5 m²/cm³); water permeability (1-10 ml cm²/min @80-120 mm Hg preferable, most preferable <5 ml cm²/min @120 mmHg); and orientation of polymer/fibers (allows proper cell seeding, adherence, growth, and ECM formation). Polymer/fiber orientation will also allow proper cell migration, and is important for the minced tissue fragments such that cells will migrate out of the fragments and populate the TEBV.

The TEBV has biocompatibility that reflects desired properties for a TEBV that, in conjunction with one or more of cells, cell sheets, cell lysate, minced tissue, and a bioreactor process, will replace a small diameter, damaged or diseased vein or artery blood vessel. Desirable biocompatibility includes but is not limited: absorption (6-24 months preferable to allow greatest vol. of TEBV to be occupied by cells and ECM); tissue reaction (minimal); cell compatibility (adherence, viability, growth, migration and differentiation not negatively impacted by TEBV); residual solvent (minimal); residual EtO (minimal); and hemocompatible (non-thrombogenic).

The tissue engineered blood vessel scaffold is prepared by the following method:

A first braided mesh tube having an inner surface and an outer surface is provided as described above and placed on a mandrel. Then, a melt blown sheet is provided as described above and rolled onto the outer surface of the first braided mesh tube. Next, a second braided mesh tube is positioned over the rolled melt blown sheet.

In one embodiment, the tissue engineered blood vessel further comprises cells. Suitable cells that may be combined with the TEBV include, but are not limited to: stem cells such as multipotent or pluripotent stem cells; progenitor cells, such as smooth muscle progenitor cells and vascular endothelium progenitor cells; embryonic stem cells; postpartum tissue derived cells such as, placental tissue derived cells and umbilical tissue derived cells; endothelial cells, such as vascular endothelial cells; smooth muscle cells, such as vascular smooth muscle cells; precursor cells derived from adipose tissue; and arterial cells, such as cells derived from the radial artery and the left and right internal mammary artery (IMA), also known as the internal thoracic artery.

In one embodiment, the cells are human umbilical tissue derived cells (hUTCs). The methods for isolating and collecting human umbilical tissue-derived cells (hUTCs) (also referred to as umbilical-derived cells (UDCs)) are described in U.S. Pat. No. 7,510,873, incorporated herein by reference in its entirety. In another embodiment, the TEBV further comprises human umbilical tissue derived cells (hUTCs) and one or more other cells. The one or more other cells includes, but is not limited to vascular smooth muscle cells (SMCs), vascular smooth muscle progenitor cells, vascular endothelial cells (ECs), or vascular endothelium progenitor cells, and/or other multipotent or pluripotent stem cells. hUTCs in combination with one or more other cells on the TEBV may enhance the seeding, attachment, and proliferation of, for example, ECs and SMCs on the TEBV. hUTCs may also promote the differentiation of the EC or SMC or progenitor cells in the TEBV construct. This may promote the maturation of TEBVs during the in vitro culture as well as the engraftment during the in vivo implantation. hUTCs may provide trophic support or provide and enhance the expression of ECM proteins. The trophic effects of the cells, including hUTCs, can lead to proliferation of the vascular smooth muscle or vascular endothelium of the patient. The trophic effects of the cells, including hUTCs, may induce migration of vascular smooth muscle cells, vascular endothelial cells, skeletal muscle progenitor cells, vascular smooth muscle progenitor cells, or vascular endothelium progenitor cells to the site or sites of the regenerated blood vessel.

Cells can be harvested from a patient (before or during surgery to repair the tissue) and the cells can be processed under sterile conditions to provide a specific cell type. One of skill in the art is aware of conventional methods for harvesting and providing the cells as described above such as described in Osteoarthritis Cartilage 2007 February; 15(2):226-31 and incorporated herein by reference in their entirety. In another embodiment the cells are genetically modified to express genes of interest responsible for pro-angiogenic activity, anti-inflammatory activity, cell survival, cell proliferation or differentiation or immunomodulation.

The cells can be seeded on the TEBV for a short period of time, e.g. less than one day, just prior to implantation, or cultured for longer a period, e.g. greater than one day, to allow for cell proliferation and extracellular matrix synthesis within the seeded TEBV prior to implantation. In one embodiment, a single cell type is seeded on the TEBV. In another embodiment, one or more cell types are seeded on the TEBV. Various cellular strategies could be used with these scaffolds (i.e., autologous, allogenic, xenogeneic cells etc.). In one embodiment, smooth muscle cells can be seeded on the outer lumen of the TEBV and in another embodiment, endothelial cells can be seeded in the inner lumen of the TEBV. The cells are seeded in an amount sufficient to provide a confluent cell layer. Preferably, cell seeding density is about 2×10⁵/cm².

In another embodiment the tissue engineered blood vessel further comprises cell sheets. Cell sheets may be made of hUTCs or other cell types. Methods of making cell sheets are described in U.S. application Ser. No. 11/304,091, published on Jul. 13, 2006 as U.S. Patent Publication No. US 2006-0153815 A1 and incorporated herein by reference in its entirety. The cell sheet is generated using thermoresponsive polymer coated dishes that allow harvesting intact cell sheets with the decrease of the temperature. Alternatively, other methods of making cell sheets include, but are not limited to growing cells in a form of cell sheets on a polymer film. Selected cells may be cultured on a surface of glass, ceramic or a surface-treated synthetic polymer. For example, polystyrene that has been subjected to a surface treatment, like gamma-ray irradiation or silicon coating, may be used as a surface for cell culture. Cells grown to over 85 percent confluence form cell sheet layer on cell growth support device. Cell sheet layer may be separated from cell growth support device using proteolysis enzymes, such as trypsin or dispase. Non-enzymatic cell dissociation could also be used. A non-limiting example includes a mixture of chelators sold under the trade name CELLSTRIPPER (Mediatech, Inc., Herndon, Va.), a non-enzymatic cell dissociation solution designed to gently dislodge adherent cells in culture while reducing the risk of damage associated with enzymatic treatments.

Alternatively, the surface of the cell growth support device, from which cultured cells are collected, may be a bed made of a material from which cells detach without a proteolysis enzyme or chemical material. The bed material may comprise a support and a coating thereon, wherein the coating is formed from a polymer or copolymer which has a critical solution temperature to water within the range of 0° C. to 80° C.

In one embodiment, one or more cells sheets are combined with the TEBV as described herein above by layering the cell sheets on the melt blown sheet and then rolling the sheet on the tube. The one or more cell sheets may be of the same cell type or of different cell types as described herein above. In one embodiment, multiple cell sheets could be combined to form a robust vascular construct. For example, cell sheets made of endothelial cells and smooth muscle cells could be combined with the scaffold to form TEBVs. Alternatively, other cell types such as hUTC cell sheets could be combined with endothelial cell sheets and the scaffold to form TEBVs. Furthermore, cell sheets made of hUTCs can be wrapped around a pre-formed TEBV composed of a scaffold, ECs, and SMCs to provide trophic factors supporting maturation of the construct.

Cell sheets may be grown on the melt blown sheet to provide reinforcement and mechanical properties to the cell sheets. Reinforced cell sheets can be formed by placing biodegradable or non-biodegradable reinforcing members at the bottom of support device prior to seeding support device with cells. Reinforcing members are as described herein above. Cell sheet layer that results will have incorporated the reinforcing scaffold providing additional strength to the cell sheet layer, which can be manipulated without the requirement for a backing layer. A preferred reinforcing scaffold is a mesh comprised of poly(p-dioxanone). The mesh can be placed at the bottom of a Corning® Ultra low attachment dish. Cells can then be seeded on to the dishes such that they will form cell-cell interactions but also bind to the mesh when they interact with the mesh. This will give rise to reinforced cell sheets with better strength and handling characteristics. Such reinforced cell sheets may be rolled into a TEBV or the reinforced cell sheet layer may be disposed on a scaffold (as described above).

In another embodiment, the cell sheet is genetically engineered. The genetically engineered cell sheet comprises a population of cells wherein at least one cell of the population of cells is transfected with an exogenous polynucleotide such that the exogenous polynucleotide expresses express diagnostic and/or therapeutic product (e.g., a polypeptide or polynucleotide) to assist in tissue healing, replacement, maintenance and diagnosis. Examples of “proteins of interest” (and the genes encoding same) that may be employed herein include, without limitation, cytokines, growth factors, chemokines, chemotactic peptides, tissue inhibitors of metalloproteinases, hormones, angiogenesis modulators either stimulatory or inhibitory, immune modulatory proteins, neuroprotective and neuroregenerative proteins and apoptosis inhibitors. More specifically, preferred proteins include, without limitation, erythropoietin (EPO), EGF, VEGF, FGF, PDGF, IGF, KGF, IFN-α, IFN-δ, MSH, TGF-α, TGF-β, TNF-α, IL-1, BDNF, GDF-5, BMP-7 and IL-6.

In another embodiment the tissue engineered blood vessel further comprises cell lysate. Cell lysates may be obtained from cells including, but not limited to stem cells such as multipotent or pluripotent stem cells; progenitor cells, such as smooth muscle progenitor cells and vascular endothelium progenitor cells; embryonic stem cells; postpartum tissue derived cells such as, placental tissue derived cells and umbilical tissue derived cells, endothelial cells, such as vascular endothelial cells; smooth muscle cells, such as vascular smooth muscle cells; precursor cells derived from adipose tissue; and arterial cells such as cells derived from the radial artery and the left and right internal mammary artery (IMA), also known as the internal thoracic artery. The cell lysates and cell soluble fractions may be stimulated to differentiate along a vascular smooth muscle or vascular endothelium pathway. Such lysates and fractions thereof have many utilities. Use of lysate soluble fractions (i.e., substantially free of membranes) in vivo, for example, allows the beneficial intracellular milieu to be used allogeneically in a patient without introducing an appreciable amount of the cell surface proteins most likely to trigger rejection or other adverse immunological responses.

Methods of lysing cells are well-known in the art and include various means of mechanical disruption, enzymatic disruption, chemical disruption, or combinations thereof. Such cell lysates may be prepared from cells directly in their growth medium and thus containing secreted growth factors and the like, or may be prepared from cells washed free of medium in, for example, PBS or other solution. The cell lysate can be used to create a TEBV according to the present invention by placing a TEBV into a cell culture plate and adding cell lysate supernatant onto the TEBV. The lysate loaded TEBV can then be placed into a lyophilizer for lyophilization.

In yet another embodiment the tissue engineered blood vessel further comprises minced tissue. Minced tissue has at least one viable cell that can migrate from the tissue fragments onto the TEBV. More preferably, the minced tissue contains an effective amount of cells that can migrate from the tissue fragments and begin populating the TEBV. Minced tissue may be obtained from one or more tissue sources or may be obtained from one source. Minced tissue sources include, but are not limited to muscle tissue, such as skeletal muscle tissue and smooth muscle tissue; vascular tissue, such as venous tissue and arterial tissue; skin tissue, such as endothelial tissue; and fat tissue.

The minced tissue is prepared by first obtaining a tissue sample from a donor (autologous, allogenic, or xenogeneic) using appropriate harvesting tools. The tissue sample is then finely minced and divided into small fragments either as the tissue is collected, or alternatively, the tissue sample can be minced after it is harvested and collected outside the body. In embodiments where the tissue sample is minced after it is harvested, the tissue samples can be washed three times in phosphate buffered saline. The tissue can then be minced into small fragments in the presence of a small quantity, for example, about 1 ml, of a physiological buffering solution, such as, phosphate buffered saline, or a matrix digesting enzyme, such as 0.2 percent collagenase in Ham's F12 medium. The tissue is minced into fragments of approximately 0.1 to 1 mm³ in size. Mincing the tissue can be accomplished by a variety of methods. In one embodiment, the mincing is accomplished with two sterile scalpels cutting in parallel and opposing directions, and in another embodiment, the tissue can be minced by a processing tool that automatically divides the tissue into particles of a desired size. In one embodiment, the minced tissue can be separated from the physiological fluid and concentrated using any of a variety of methods known to those having ordinary skill in the art, such as, for example, sieving, sedimenting or centrifuging. In embodiments where the minced tissue is filtered and concentrated, the suspension of minced tissue preferably retains a small quantity of fluid in the suspension to prevent the tissue from drying out.

The suspension of minced living tissue can be used to create a TEBV according to the present invention by depositing the suspension of living tissue upon a biocompatible TEBV, such that the tissue and the TEBV become associated. Preferably, the tissue is associated with at least a portion of the TEBV. The TEBV can be implanted in a subject immediately, or alternatively, the construct can be incubated under sterile conditions that are effective to maintain the viability of the tissue sample.

In another aspect of the invention, the minced tissue could consist of the application of two distinct minced tissue sources (e.g., one surface could be loaded with minced endothelial tissue and the other surface could be loaded with minced smooth muscle tissue).

In one embodiment, the tissue engineered blood vessels and one or more of cells, cell sheets, cell lysate, or minced tissue is enhanced by combining with bioactive agents. Suitable bioactive agents include, but are not limited to an antithrombogenic agent, an anti-inflammatory agent, an immunosuppressive agent, an immunomodulatory agent, pro-angiogenic, an antiapoptotic agent, antioxidants, growth factors, angiogenic factors, myoregenerative or myoprotective drugs, conditioned medium, extracellular matrix proteins, such as, collagen, atelocollagen, laminin, fibronectin, vitronectin, tenascin, integrins, glycosaminoglycans (hyaluronic acid, chondroitin sulfate, dermatan sulfate, heparan sulfate, heparin, keratan sulfate and the like), elastin and fibrin; growth factors and/or cytokines, such as vascular endothelial cell growth factors, platelet derived growth factors, epidermal growth factors, fibroblast growth factors, hepatocyte growth factors, insulin-like growth factors, and transforming growth factors.

Conditioned medium from cells as described previously herein allows the beneficial trophic factors secreted by the cells to be used allogeneically in a patient without introducing intact cells that could trigger rejection, or other adverse immunological responses. Conditioned medium is prepared by culturing cells in a culture medium, then removing the cells from the medium. Conditioned medium prepared from populations of cells, including hUTCs, may be used as is, further concentrated, for example, by ultrafiltration or lyophilization, or even dried, partially purified, combined with pharmaceutically-acceptable carriers or diluents as are known in the art, or combined with other bioactive agents. Conditioned medium may be used in vitro or in vivo, alone or combined with autologous or allogenic live cells, for example. The conditioned medium, if introduced in vivo, may be introduced locally at a site of treatment, or remotely to provide needed cellular growth or trophic factors to a patient. This same medium may also be used for the maturation of the TEBVs. Alternatively, hUTC or other cells conditioned medium may also be lyophilized onto the TEBVs prior to seeding with both ECs and SMCs.

From a manufacturing perspective, hUTCs or other cells or conditioned medium may shorten the time for the in vitro culture or fabrication of TEBVs. This will also result in the use of less starting cells making autologous sources of ECs and SMCs a more viable option.

In one embodiment, the tissue engineered blood vessels further comprising cells, cell sheets, cell lysate, or minced tissue is enhanced by combining with a bioreactor process. These tissue engineered blood vessels may be cultured with or without a bioreactor process. The TEBV may be cultured using various cell culture bioreactors, including but not limited to a spinner flask, a rotating wall vessel (RWV) bioreactor, a perfusion-based bioreactor or combination thereof. In one embodiment the cell culture bioreactor is a rotating wall vessel (RWV) bioreactor or a perfusion-based bioreactor. The perfusion-based bioreactor will consist of a device for securing the TEBV and allow culture medium to flow through the lumen of the TEBV, and may also allow for seeding and culturing of cells on both the inner (lumen) and outer surfaces of the TEBV. The perfusion bioreactors may also have the capability of generating pulsatile flow and various pressures for conditioning of the cell-seeded TEBV prior to implantation. Pulsatile flow stress during bioreactor process is preferably 1-25 dynes/cm² over 1 day-1 yr, and more preferably a gradual increase from 1-25 dynes/cm² over 2-4 wks.

The TEBV having cells, cell sheets, cell lysate, or minced tissue and optionally bioactive agents may be cultured for longer a period, e.g. greater than one day, to allow for cell proliferation and matrix synthesis within the TEBV prior to implantation. Cell sheets, cell lysate, or minced tissue are applied to the TEBV as described herein above and transferred to the bioreactor for longer term culture, or more preferably, seeded and cultured within the bioreactor. Multiple bioreactors may be also used sequentially, e.g. one for initial seeding of cells, and another for long-term culture.

The process of seeding and culturing cells on the TEBV using a bioreactor may be repeated with multiple cell types sequentially, e.g. smooth muscle cells are seeded and cultured for a period of time, followed by seeding and culture of endothelial cells, or simultaneously (e.g. smooth muscle cells on the outer surface, and endothelial cells with on the inner surface (lumen) of the scaffolds). The TEBV may or may not be cultured for a period of time to promote maturation. The bioreactor conditions can be controlled as to promote proper maturation of the construct. Following the culture period, the construct can be removed and implanted into a vascular site in an animal or human.

General cell culture conditions include temperatures of 37° C. and 5 percent CO₂. The cell seeded constructs will be cultured in a physiological buffered salt solution maintained at or near physiological pH. Culture media can be supplemented with oxygen to support metabolic respiration. The culture media may be standard formulations or modified to optimally support cell growth and maturation in the construct. The culture media may contain a buffer, salts, amino acids, glucose, vitamins and other cellular nutrients. The media may also contain growth factors selected to establish endothelial and smooth muscle cells within the construct. Examples of these may include VEGF, FGF2, angiostatin, endostatin, thrombin and angiotensin II. The culture media may also be perfused within the construct to promote maturation of the construct. This may include flow within the lumen of the vessel at pressures and flow rates that may be at or near values that the construct may be exposed to upon implant.

The media is specific for the cell type being cultured (i.e., endothelial medium for endothelial cells, and smooth muscle cell medium for SMCs). For the perfusion bioreactor especially, there are other considerations taken into account such as but not limited to shear stress (related to flow rate), oxygen tension, and pressure.

The TEBVs can be also be electrically stimulated to enhance the attachment or proliferation of the different cell types. The electrical stimulation can be performed during the culture and expansion of the cells prior to the fabrication of the TEBV, during the maturation phase of the TEBV, or during implantation. Cells, including hUTCs may also be electrically stimulated during the production of conditioned medium.

The present invention also provides a method for the repair or regeneration of tissue inserting the TEBV described above at a location on the blood vessel in need of repair. These TEBV structures are particularly useful for the regeneration of tissue between two or more different types of tissues. For a multi-cellular system in the simplest case, one cell type could be present on one side of the scaffold and a second cell type on the other side of the scaffold. Examples of such regeneration can be vascular tissue with smooth muscle on the outside and endothelial cells on the inside to regenerate vascular structures. This process can be achieved by culturing different cell types on either side of the melt blown sheet at the same time or in a step wise fashion.

The invention also relates to methods of treating tissue using the TEBV prepared by the methods described herein. The TEBV can be used in arteriovenous grafting, coronary artery grafting or peripheral artery grafting. For example, in a typical arteriovenous (AV) surgical procedure used for the treatment of end-stage renal failure patients, the surgeon makes an incision through the skin and muscle of the forearm. An artery and a vein are selected (usually the radial artery and the cephalic vein) and an incision is made into each. The TEBV is then used to anastomos the ends of the artery and the vein. The muscle and skin are then closed. After the graft has properly healed (4-6 weeks), the successful by-pass can be used to treat the patient's blood.

In a coronary by-pass (CABG) procedure, a TEBV would be used for patients suffering from arteriosclerosis, a common arterial disorder characterized by arterial walls that have thickened, have lost elasticity, and have calcified. This leads to a decrease in blood supply which can lead to damage to the heart, stroke and heart attacks. In a typical CABG procedure, the surgeon opens the chest via a sternotomy. The heart's functions are taken over by a Heart and Lung machine. The diseased artery is located and one end of the TEBV is sewn onto the coronary arteries beyond the blockages and the other end is attached to the aorta. The heart is restarted, the sternum is wired together and the incisions are sutured closed. Within a few weeks, the successful by-pass procedure is fully healed and the patient is functioning normally.

The following examples are illustrative of the principles and practice of this invention, although not limited thereto. Numerous additional embodiments within the scope and spirit of the invention will become apparent to those skilled in the art once having the benefit of this disclosure.

### Example 1

### Synthesis of Segmented p-Dioxanone-Rich Poly(epsilon-caprolactone-co-p-dioxanone) Triblock Copolymer at 17/83 by Mole

Using a 10-gallon stainless steel oil jacketed reactor equipped with agitation, 4,123 grams of epsilon-caprolactone was added along with 63.9 grams of diethylene glycol and 16.6 mL of a 0.33M solution of stannous octoate in toluene. After the initial charge, a purging cycle with agitation at a rotational speed of 6 RPM in an upward direction was conducted. The reactor was evacuated to pressures less than 550 mTorr followed by the introduction of nitrogen gas. The cycle was repeated once again to ensure a dry atmosphere. At the end of the final nitrogen purge, the pressure was adjusted to be slightly above one atmosphere. The vessel was heated by setting the oil controller at 195° C. at a rate of 180° C. per hour. The reaction continued for 6 hours and 10 minutes from the time the oil temperature reached 195° C.

In the next stage, the oil controller set point was decreased to 120° C., and 20,877 grams of molten p-dioxanone monomer was added from a melt tank with the agitator speed of 7 RPM in an upward direction for 70 minutes. At the end of the reaction, the agitator speed was reduced to 5 RPM, and the polymer was discharged from the vessel into suitable containers. The containers were placed into a nitrogen oven set at 80° C. for a period of 4 days. During this solid state polymerization step, the constant nitrogen flow was maintained in the oven to reduce possible moisture-induced degradation.

The crystallized polymer was then removed from the containers and placed into a freezer set at approximately −20° C. for a minimum of 24 hours. The polymer was then removed from the freezer and placed into a Cumberland granulator fitted with a sizing screen to reduce the polymer granules to approximately 3/16 inches in size. The granules were then sieved to remove any “fines” and weighed. The net weight of the ground and sieved polymer was 19.2 kg, which was next placed into a 3 cubic foot Patterson-Kelley tumble dryer to remove any residual monomer. The dryer was closed and the pressure was reduced to less than 200 mTorr. Once the pressure was below 200 mTorr, dryer rotation was activated at a rotational speed of 5-10 RPM with no heat for 10 hours. After 10 hours, the oil temperature was set to 80° C. at a heat up rate of 120° C. per hour. The oil temperature was maintained at approximately 80° C. for a period of 32 hours. At the end of the heating period, the batch was allowed to cool for a period of 3 hours while maintaining rotation and vacuum. The polymer was discharged from the dryer by pressurizing the vessel with nitrogen, opening the discharge valve, and allowing the polymer granules to descend into waiting vessels for long term storage.

The long term storage vessels were air tight and outfitted with valves allowing for evacuation so that the resin was stored under vacuum. The dried resin exhibited an inherent viscosity of 1.1 dL/g, as measured in hexafluoroisopropanol at 25° C. and at a concentration of 0.10 g/dL. Gel permeation chromatography analysis showed a weight average molecular weight of approximately 43,100 Daltons. Nuclear magnetic resonance analysis confirmed that the resin contained 83.0 mole percent poly(p-dioxanone) and 16.2 mole percent poly(epsilon-caprolactone) with a residual monomer content of less than 1.0 percent.

### Example 2

### Synthesis of Segmented p-Dioxanone-Rich Poly(epsilon-caprolactone-co-p-dioxanone) Triblock Copolymer at 9/91 by Mole (PDO-Rich Cap/PDO copolymer)

Using a 10-gallon stainless steel oil jacketed reactor equipped with agitation, 2,911 grams of epsilon-caprolactone was added along with 90.2 grams of diethylene glycol and 23.4 mL of a 0.33M solution of stannous octoate in toluene. The reaction conditions in the first stage were closely matched those in Example 1.

In the second, copolymerization stage, the oil controller set point was decreased to 120° C., and 32,089 grams of molten p-dioxanone monomer was added from a melt tank with the agitator rotating at 7.5 RPM in a downward direction for 40 minutes. The oil controller was then set to 115° C. for 20 minutes, then to 104° C. for one hour and 45 minutes, and finally to 115° C. 15 minutes prior to the discharge. The post curing stage (80° C./4 days) and grounding and sieving procedure were conducted according to Example 1. The net weight of the ground and sieved polymer was 31.9 kg, which was then placed into a 3 cubic foot Patterson-Kelley tumble dryer for monomer removal following conditions described in the Example 1.

The dried resin exhibited an inherent viscosity of 0.97 dL/g, as measured in hexafluoroisopropanol at 25° C. and at a concentration of 0.10 g/dL. Gel permeation chromatography analysis showed a weight average molecular weight of approximately 33,000 Daltons. Nuclear magnetic resonance analysis confirmed that the resin contained 90.4 mole percent poly(p-dioxanone) and 8.7 mole percent poly(epsilon-caprolactone) with a residual monomer content of less than 1.0 percent.

### Example 3

### Synthesis of Segmented epsilon-caprolactone-Rich Poly(epsilon-caprolactone-co-p-dioxanone) Triblock Copolymer at 91/9 by Mole (Cap-Rich Cap/PDO copolymer) [Initial Feed Charge of 75/25 Cap/PDO]

Using a 10-gallon stainless steel oil jacketed reactor equipped with agitation, 18,492 grams of epsilon-caprolactone was added along with 19.1 grams of diethylene glycol and 26.2 mL of a 0.33M solution of stannous octoate in toluene. After the initial charge, a purging cycle with agitation at a rotational speed of 10 RPM in a downward direction was initiated. The reactor was evacuated to pressures less than 500 mTorr followed by the introduction of nitrogen gas. The cycle was repeated once again to ensure a dry atmosphere. At the end of the final nitrogen purge, the pressure was adjusted to be slightly above one atmosphere. The rotational speed of the agitator was reduced to 7 RPM in a downward direction. The vessel was heated by setting the oil controller at 195° C. at a rate of 180° C. per hour. The reaction continued for 4 hours from the time the oil temperature reached 195° C. After this period, the reaction was continued for an additional ½ hour under vacuum to remove the unreacted epsilon-caprolactone monomer.

In the second, copolymerization stage, the oil controller set point was decreased to 180° C., and 5,508 grams of molten p-dioxanone monomer was added from a melt tank with the agitator speed of 10 RPM in a downward direction for 15 minutes. The agitator speed was then reduced to 7.5 RPM in the downward direction. The oil controller was then set up to 150° C. for 30 minutes, then to 115° C. for one hour and 15 minutes, then to 110° C. for 20 minutes, and finally to 112° C. for 30 minutes 15 minutes prior to the discharge.

At the end of the final reaction period, the agitator speed was reduced to 2 RPM in the downward direction, and the polymer was discharged from the vessel into suitable containers. Upon cooling, the polymer was removed from the containers and placed into a freezer set at approximately −20° C. for a minimum of 24 hours. The polymer was then removed from the freezer and placed into a Cumberland granulator fitted with a sizing screen to reduce the polymer granules to approximately 3/16 inches in size. The granules were sieved to remove any “fines” and weighed. The net weight of the ground and sieved polymer was 17.5 kg, which was then placed into a 3 cubic foot Patterson-Kelley tumble dryer to remove any residual monomer.

The dryer was closed, and the pressure was reduced to less than 200 mTorr. Once the pressure was below 200 mTorr, dryer rotation was activated at a rotational speed of 5-10 RPM with no heat for 10 hours. After the 10 hour period, the oil temperature was set to 40° C. at a heat up rate of 120° C. per hour. The oil temperature was maintained at 40° C. for a period of 32 hours. At the end of this heating period, the batch was allowed to cool for a period of 4 hours while maintaining rotation and vacuum. The polymer was discharged from the dryer by pressurizing the vessel with nitrogen, opening the discharge valve, and allowing the polymer granules to descend into waiting vessels for long term storage.

The long term storage vessels were air tight and outfitted with valves allowing for evacuation so that the resin was stored under vacuum. The dried resin exhibited an inherent viscosity of 2.01 dL/g, as measured in hexafluoroisopropanol at 25° C. and at a concentration of 0.10 g/dL. Gel permeation chromatography analysis showed a weight average molecular weight of approximately 71,000 Daltons. Nuclear magnetic resonance analysis confirmed that the resin contained 8.61 mole percent poly(p-dioxanone) and 90.88 mole percent poly(epsilon-caprolactone) with a residual monomer content of less than 1.0 percent.

### Example 4

### Melt Blown Nonwoven Made from 9/91 Cap/PDO Copolymer

On a six-inch melt blown nonwoven line of the type described hereinabove equipped with single screw extruder, a copolymer of 9/91 Cap/PDO (prepared as described in Example 2) with 33,000 Daltons weight-average molecular weight was extruded into melt blown nonwovens. This process involved feeding the solid polymer pellets into a feeding hopper on an extruder. The extruder had a 1¼″ single screw with three heating zones which gradually melt the polymer and extruded the molten polymer through a connector or transfer line. Finally, the molten polymer was pushed into a die assembly containing many capillary holes of which emerged small diameter fibers. The fiber diameter was attenuated at the die exit as the fiber emerged using high velocity hot air. About 6 inches from the die exit was a rotating collection drum on which the fibrous web was deposited and conveyed to a wind up spool. The melt blown line was of standard design as described by Buntin, Keller and Harding in U.S. Pat. No. 3,978,185, the contents of which are hereby incorporated by reference in their entirety. The die used had 210 capillary holes with a diameter of 0.018 inch per hole. The processing conditions and resulting properties of melt blown nonwovens are listed in the following table which follows:

Experimental conditions for Melt-Blown
processing of 9/91 Cap/PDO copolymer

| Samples | 1 | 2 | 3 |
|---|---|---|---|
| Processing Conditions: | | | |
| Die Temperature (° C.) | 184 | 183 | 182 |
| Die Pressure (psi) | 400 | 400 | 400 |
| Air Temperature (° C.) | 255 | 255 | 255 |
| Air Pressure (psi) | 16 | 16 | 16 |
| Metering Pump Speed (rpm) | 2.3 | 2.3 | 2.3 |
| Throughput (grams/hole/minute) | 0.161 | 0.161 | 0.161 |
| Collector Speed (meters/minute) | 2.70 | 5.49 | 10.98 |
| Nonwoven Properties: | | | |
| Base Weight (gsm) | 40 | 20 | 10 |
| Fiber Diameter (micrometres) | 3.0-6.0 | 3.0-6.0 | 3.0-6.0 |
| Average Pore Size (micrometres) | 26.5 | 35.7 | 44.1 |

### Example 5

### Melt Blown Nonwoven Made from 17/83 Cap/PDO Copolymer

On a six-inch melt blown nonwoven line of the type described hereinabove, equipped with single screw extruder, a copolymer of Cap/PDO 17/83 (prepared as described in Example 1) with 43,100 Daltons weight-average molecular weight was extruded into melt blown nonwovens. This process involved feeding the solid polymer pellets into a feeding hopper on an extruder. The extruder had a 1¼″ single screw with three heating zones which gradually melt the polymer and extruded the molten polymer through a connector or transfer line. Finally, the molten polymer was pushed into a die assembly containing many capillary holes of which emerged small diameter fibers. The fiber diameter was attenuated at the die exit as the fiber emerges using high velocity hot air. About 6 inches from the die exit was a rotating collection drum on which the fibrous web was deposited and conveyed to a wind up spool. The melt blown line was of standard design as described by Buntin, Keller and Harding in U.S. Pat. No. 3,978,185, the contents of which are hereby incorporated by reference in their entirety. The die used had 210 capillary holes with a diameter of 0.018 inch per hole. Similar processing conditions as in the previous example of Cap/PDO 10/90 were used to make the nonwoven. Cap/PDO 17/83, however, was too elastic and stretchy. In addition, Cap/PDO 17/83 solidified too slowly to form fibrous shapes for melt blown nonwovens. It either formed very big size of fibers and/or granulated particles. Thus, the experiment indicated Cap/PDO 17/83 is not suitable for making melt blown nonwovens.

### Example 6A

### Melt Blown Nonwoven Made from 25/75 Epsilon-Caprolactone/Glycolide Copolymer

This example illustrates the processing of an epsilon-caprolactone/glycolide 25/75 copolymer (final mole composition) into melt blown nonwoven constructs. The copolymer used in this example can be made by the method outlined in the paper entitled, “Monocryl® suture, a new ultra-pliable absorbable monofilament suture” Biomaterials, Volume 16, Issue 15, October 1995, Pages 1141-1148.

On a six-inch melt blown nonwoven line equipped with single screw extruder, the epsilon-caprolactone/glycolide copolymer having a composition of 25 mole percent polymerized epsilon-caprolactone and 75 mole percent of polymerized glycolide, and having an inherent viscosity (IV) of 1.38 dL/g, was extruded into melt blown nonwoven constructs. The melt blown line was of standard design as described by Buntin, Keller and Harding in U.S. Pat. No. 3,978,185.

The process employed involved feeding the solid polymer pellets into a feeding hopper on extruder. The extruder was equipped with a 1¼″ diameter single screw with three heating zones. The extruder gradually rendered the polymer molten and conveyed the melt through a connector or transfer line. Finally, the molten polymer was pushed into a die assembly containing many capillary holes (arranged in the traditional linear fashion) through which emerged small diameter fibers. The fiber diameter was attenuated using high velocity hot air at the die exit as the fibers emerged. The fibrous web ensuing from the die assembly was deposited on a rotating collection drum positioned about 6 inches from the die exit. The web then conveyed onto a wind up spool. The die used had 210 capillary holes with a diameter of 0.014 inch per hole. The processing conditions and resulted properties of the melt blown nonwoven constructs are listed in the following Table 1.

TABLE 1
Processing Conditions and Resulted
Melt Blown Nonwoven Properties.

| Samples | 1 | 2 |
|---|---|---|
| Processing Conditions: | | |
| Die Temperature (° C.) | 237 | 236 |
| Die Pressure (psi) | 350 | 350 |
| Air Temperature (° C.) | 270 | 270 |
| Air Pressure (psi) | 17 | 17 |
| Extruder Speed (rpm) | 8.1 | 8.1 |
| Throughput (grams/hole/minute) | 0.188 | 0.188 |
| Collector Speed (meters/minute) | 4.2 | 8.0 |
| Nonwoven Properties: | | |
| Base Weight (gsm) | 38 | 20 |
| Fiber Diameter (micrometres) | 2.5-6.0 | 2.5-6.0 |
| Average Pore Size (micrometres) | 19.9 | 30.5 |

### Example 6B

### Melt Blown Nonwoven Made from Poly(p-Dioxanone)Homopolymer

The Poly(p-Dioxanone) homopolymer used in this example can be made by the methods outlined in the literature. These include the descriptions provide in the book entitled, “Handbook of biodegradable polymers”, Abraham J. Domb, Joseph Kost, David M. Wiseman, eds. (CRC Press, 1997), especially Chapter 2 “Poly(p-Dioxanone) and Its Copolymers” authored by R. S. Bezwada, D. D. Jamiolkowski, and K. Cooper.

On a six-inch melt blown nonwoven line of the type described hereinabove, equipped with single screw extruder, a poly(p-dioxanone) homopolymer with 70,000 grams/mole weight-average molecular weight was extruded into melt blown nonwovens. This process involved feeding the solid polymer pellets into a feeding hopper on an extruder. The extruder had a 1¼″ single screw with three heating zones which gradually melt the polymer and extruded the molten polymer through a connector or transfer line. Finally, the molten polymer was pushed into a die assembly containing many capillary holes of which emerged small diameter fibers. The fiber diameter was attenuated at the die exit as the fiber emerges using high velocity hot air. About 6 inches from the die exit was a rotating collection drum on which the fibrous web was deposited and conveyed to a wind up spool. The melt blown line was of standard design as described by Buntin, Keller and Harding in U.S. Pat. No. 3,978,185, the contents of which are hereby incorporated by reference in their entirety. The die used had 210 capillary holes with a diameter of 0.018 inch per hole. The processing conditions and resulted properties of melt blown nonwovens are listed in the following Table 2.

TABLE 2
Processing Conditions and Resulted
Melt Blown Nonwoven Properties.

| Samples | 1 | 2 | 3 |
|---|---|---|---|
| Processing Conditions: | | | |
| Die Temperature (° C.) | 194 | 194 | 195 |
| Die Pressure (psi) | 600 | 600 | 600 |
| Air Temperature (° C.) | 250 | 250 | 250 |
| Air Pressure (psi) | 22 | 22 | 22 |
| Extruder Speed (rpm) | 2.3 | 2.3 | 2.3 |
| Throughput (grams/hole/minute) | 0.079 | 0.079 | 0.079 |
| Collector Speed (meters/minute) | 1.52 | 3.00 | 5.80 |
| Nonwoven Properties: | | | |
| Base Weight (gsm) | 35 | 18 | 10 |
| Fiber Diameter (micrometres) | 3.0-6.0 | 3.0-6.0 | 3.0-6.0 |
| Average Pore Size (micrometres) | 13.0 | 31.5 | 41.8 |

### Example 7

### Synthesis of a 65/35 PGA/PCL Foam Scaffold

A 5 percent wt./wt. polymer solution was prepared by dissolving 5 part 35/65 PCL/PGA with 95 parts of solvent 1,4-dioxane. The solution was prepared in a flask with a magnetic stir bar. To dissolve the copolymer completely, the mixture was gently heated to 60° C. and continuously stirred overnight. A clear homogeneous solution was then obtained by filtering the solution through an extra coarse porosity filter (Pyrex® brand extraction thimble with fritted disc).

A lyophilizer (Dura-Stop™, FTS system) was used. The freeze dryer was powered up and the shelf chamber was maintained at −17° C. for approximately 30 minutes. Thermocouples to monitor the shelf temperature were attached for monitoring. The homogeneous polymer solution was poured into an aluminum mold. The mold was placed into a lyophilizer maintained at −17° C. (pre-cooling). The lyophilization cycle was started and the shelf temperature was held at −17° C. for 15 minutes and then held at −15° C. for 120 minutes. A vacuum was applied to initiate drying of the dioxane by sublimation. The mold was cooled to −5° C. and held at this temperature for 120 minutes. The shelf temperature was raised to 5° C. and held for 120 minutes. The shelf temperature was raised again to 20° C. and held at that temperature for 120 minutes. At the end of the first lyophilization stage, the second stage of drying was begun and the shelf temperature was held at 20° C. for an additional 120 minutes. At the end of the second stage, the lyophilizer was brought to room temperature and atmospheric pressure.

### Example 8

### Attachment and Growth of Rat Smooth Muscle Cells on Poly(p-Dioxanone) Melt Blown Scaffolds and 75/25 PGA/PCL Melt Blown Scaffolds

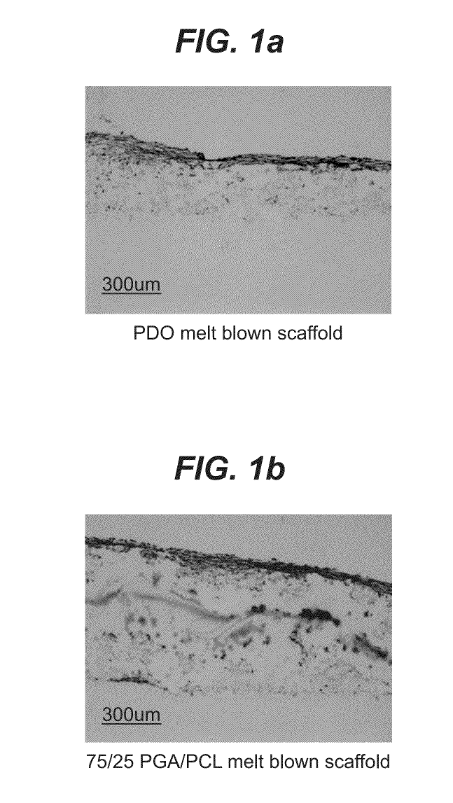

PDO melt blown scaffolds and 75/25 PGA/PCL melt blown scaffolds, prepared as described in Examples 6A and 6B above were evaluated for the growth of the Rat smooth muscle cells. Rat smooth muscle cells (SMC, Lonza Walkersville, Inc, Cat#: R-ASM-580) were suspended in SmGM-2 bulletkit (Lonza, cat #CC-3182) and then seeded onto PDO and 75/25 PGA/PCL melt blown scaffolds (5 mm diameter punches) at a density of 0.5×10⁶ cells per scaffold. The cell-seeded scaffolds were incubated at 37° C. for 2 hours prior to re-feeding the scaffolds with additional media. The scaffolds were cultured in a humidified incubator at 37° C. in an atmosphere of 5 percent CO₂ and 95 percent air and re-fed every other day. At day 1 and day 7 of culturing, the scaffolds were removed from media, washed with PBS, fixed with Live/Dead staining (Molecular Probes, Cat #L3224) and 10 percent formalin. Live/Dead stained images of both the PDO melt blown scaffolds and the 75/25 PGA/PCL melt blown scaffolds showed cell attachment and proliferation during 7 day culture period. Hematoxylin/Eosin (H&E) stained images, as shown in FIGS. 1a and 1b, showed Rat SMCs were distributed throughout the scaffolds and that these melt blown scaffolds supported cell attachment and proliferation.

### Example 9

### Attachment and Growth of Human Umbilical Tissue Cells (hUTC-on PDO Melt Blown Scaffolds and Collagen Coated PDO Melt Blown Scaffolds

PDO melt blown scaffolds (prepared as described in Example 6B) and collagen coated PDO melt blown scaffolds were evaluated for supporting human umbilical tissue cells growth. These scaffolds were punched into 5 mm diameter disks, and some of the scaffolds were coated with 25-50 ul of rat tail type 1 collagen at concentration of 50 ug/ml in 0.02N acetic acid (BD cat #354236). The coated scaffolds were incubated at room temperature for one hour and washed with PBS 3 times. The collagen coated scaffolds were allowed to air dry for half hour. Then hUTC cells, isolated and collected as described in U.S. Pat. No. 7,510,873, were seeded onto 5 mm scaffolds at a density of 0.5×106/scaffold and cultured with cell culture growth medium (DMEM/low glucose, 15 percent fetal bovine serum, glutamax solution).

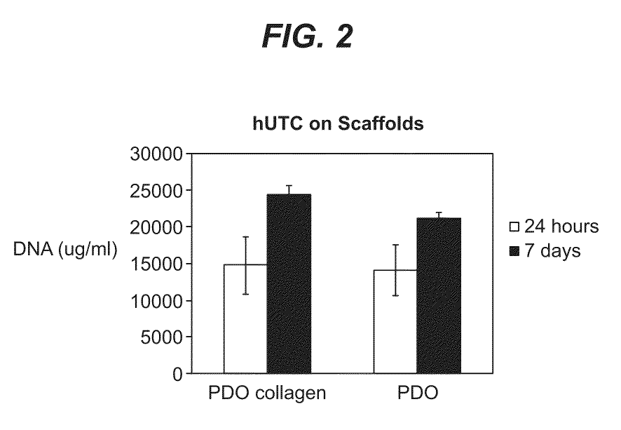

The scaffolds were harvested at 1 day and 7 days. The scaffolds with hUTC were washed with PBS once and evaluated with LIVE/DEAD staining (Molecular Probes: catalog number L-3224) and DNA measurement (CyQuant assay). The Live/Dead images and DNA results indicated that melt blown scaffolds support hUTC attachment and proliferation (FIG. 2). Some cells attached to the scaffolds at day 1, and cross section images of the scaffolds showed an increased density of cells within the scaffolds from day 1 to day 7.

### Example 10

### Preparation of Human Internal Mammary Arterial Cells

Human internal mammary artery was obtained from the National Disease Research Interchange (NDRI, Philadelphia, Pa.). To remove blood and debris, the artery was trimmed and washed in Dulbecco's modified Eagles medium or phosphate buffered saline (PBS, Invitrogen, Carlsbad, Calif.). The entire artery was then transferred to a 50 milliliter conical tube. The tissue was then digested in an enzyme mixture containing 0.25 Units/milliliter collagenase (Serva Electrophoresis, Heidelberg, Germany) and 2.5 Units/milliliter dispase (Roche Diagnostics Corporation, Indianapolis, Ind.). The enzyme mixture was then combined with iMAC growth medium (Advanced DMEM/F12 (Gibco), L-glutamine (Gibco), Pen/Strep. (Gibco) containing 10 percent fetal bovine serum (FBS). The tissue was incubated at 37° C. for two hours. The digested artery was removed from the 50 ml conical tube and discarded. The resulting digest was then centrifuged at 150 g for 5 minutes, and the supernatant was aspirated. The cell pellet resulting digest was re-suspended in 20 milliliter growth medium and filtered through a 70-micron nylon BD Falcon Cell Strainer (BD Biosciences, San Jose, Calif.). The cell suspension was centrifuged at 150 g for 5 minutes. The supernatant was aspirated and the cells were re-suspended in fresh iMAC growth medium and plated into tissue culture flask. The cells were then cultured at 37° C. and 5 percent CO₂ incubator.

### Example 11

### Attachment and Growth of Human Internal Mammary Arterial Cells (iMAC) on PDO Melt Blown Scaffolds, a 65/35 PGA/PCL Foam Scaffold, and a 90/10 PGA/PLA Needle Punched Scaffold

Three PDO melt blown scaffolds (prepared as described in Example 6B), a 65/35 PGA/PCL foam scaffold (prepared as described in Example 7), and a 90/10 PGA/PLA needle punched scaffold were evaluated for supporting human Internal Mammary Arterial cells (iMAC). The 90/10 PGA/PLA needle punched scaffold was produced by Concordia Manufacturing, LLC (Coventry, R.I.), and the thickness and density of the scaffold were 1.5 mm and 100 mg/cc.

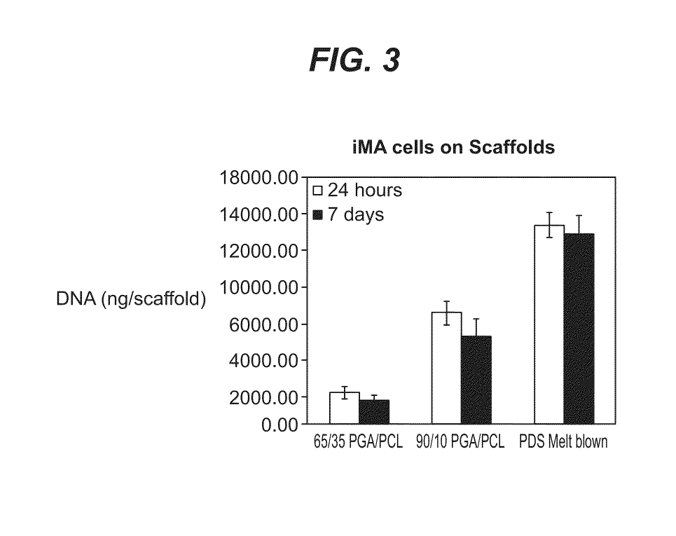

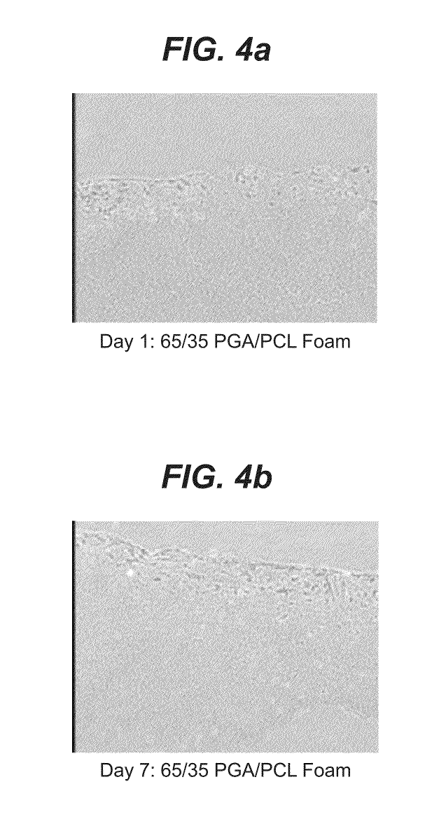

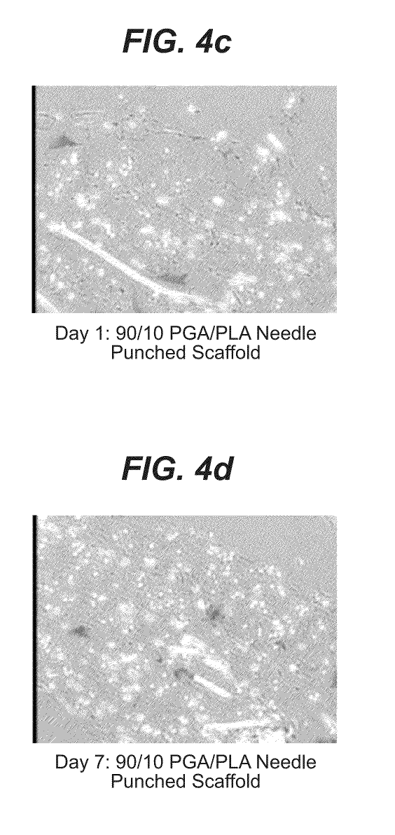

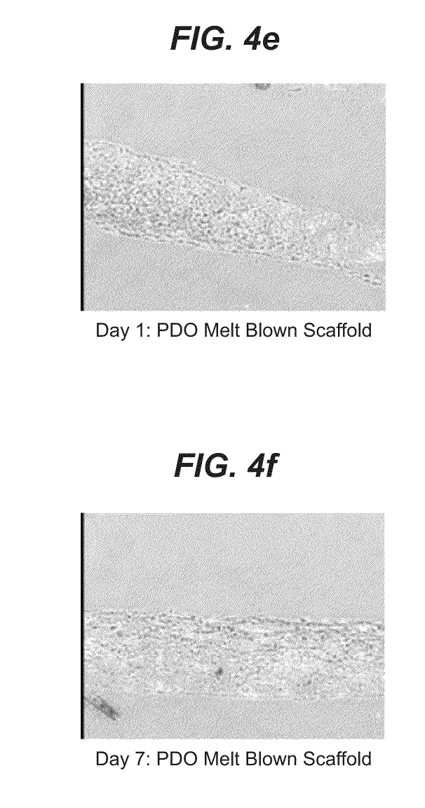

Primary iMAC cells as prepared in Example 10 were seeded onto the 65/35 PGA/PCL foam, the 90/10 PGA/PLA needle punched scaffold, and the PDO melt blown scaffolds. All the scaffolds were punched into a 5 mm diameter scaffold and seeded with iMA cells at a density of 0.5×106/scaffold and supplemented with media containing Advanced DMEM/F12 (Invitrogen Cat# 12634-010), 10 percent FBS (Gamma irradiated: Hyclone cat # SH30070.03), and Penstrep. The scaffolds were cultured for 1 day and 7 days at 37° C. and 5 percent CO2 incubator. To determine cell ingrowths, CyQuant assay (DNA content) (FIG. 3) and histology (FIGS. 4a-f) were used to measure cell adhesion and proliferation. DNA results indicated that melt blown scaffolds supported iMAC attachment and proliferation compared with the 65/35 PGA/PCL foam and the 90/10 PGA/PLA needle punched scaffolds. Histology results showed more iMAC migration into the PDO melt blown scaffold than the 65/35 PGA/PCL foam and the 90/10 PGA/PLA melt blown scaffolds at day 7.

### Example 12

### Synthesis of a Braided Mesh/Rolled Melt Blown Cap/PDO/Braided Mesh Scaffold

For the present invention, two sizes (2 mm, 3 mm) of PDO mesh tubes were fabricated at Secant Medical (Perkasie, Pa.) to form the inner and outer braided mesh tubes. Hundred micron PDO monofilament was wound onto 24 individual braiding spools and setup on one of Secant Medical's braiding machines. The 24 ends of 100 micron PDO monofilament was braided onto a 2 mm or a 3 mm mandrel having 18″ in length in a 1×1 pattern at approximately a 90° braid angle. The mandrel was then put on a rack and heat-set in an inert atmosphere oven at 85 C.° for 15 mins.

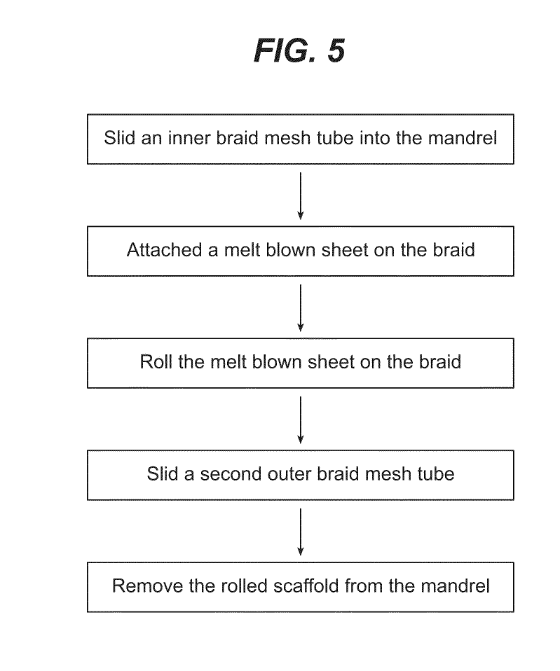

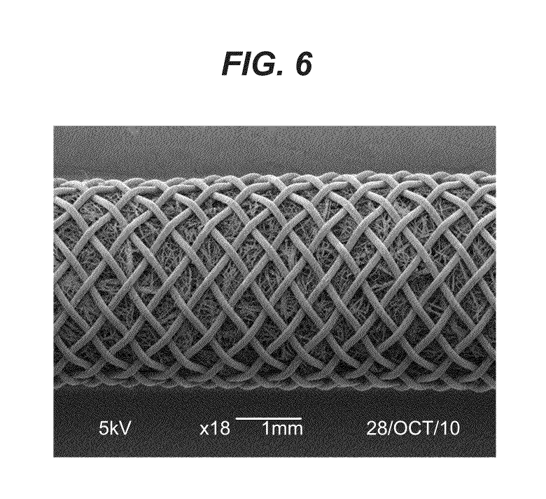

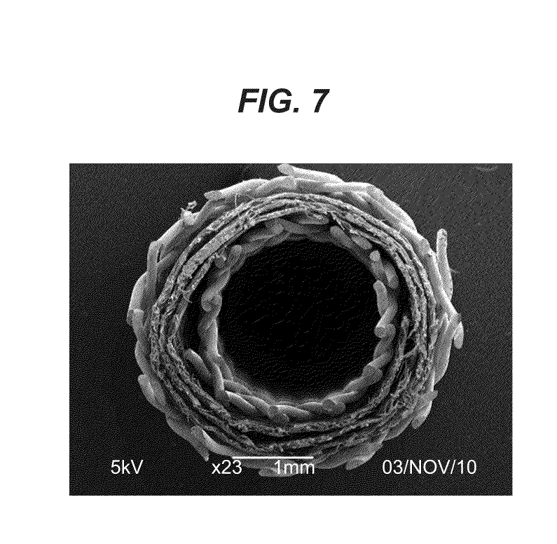

To prepare the rolled melt-blown 9/91 poly(epsilon-caprolactone-co-p-dioxanone) (9/91 Cap/PDO) sheet-mesh scaffold, a braided mesh (2 mm inner diameter, 24 ends of 100 micron polydioxanone monofilament, Secant Medical) was first compressed and placed onto a mandrel (2 mm Teflon coated rod). The braided mesh was then allowed to relax to regain its original diameter. The 9/91 Cap/PDO melt blown sheet (3 cm×3 cm sheets) was then placed onto the braided mesh and rolled. A second braided mesh (3 mm inner diameter, 24 ends of 100 micron polydioxanone monofilament, Secant Medical) was compressed and slid across the rolled melt blown tube. The second braided mesh was allowed to relax so that the mesh tightly wrapped around the rolled tube. The inner lumen mesh-rolled melt blown-outer mesh scaffold was then removed from the mandrel. FIG. 5 shows the procedure of the rolling process. FIGS. 6 and 7 show SEM images of a braided mesh/rolled melt blown Cap/PDO/braided mesh scaffold.

### Example 13

### Burst Strength Tests of Rolled Scaffolds of a Braided Mesh/a Rolled 9/91 Cap/PDO Melt Blown Tube/a Rolled Braided Mesh

Rolled scaffolds of a braided mesh/a rolled 9/91 Cap/PDO melt blown tube/a rolled braided mesh prepared as described in Example 12 above were used for testing burst strength (n=3). Three grafts were placed in complete media (DMEM low glucose supplemented with 15 percent FBS, 1 percent P/S) for a period of 1 hour before undergoing burst strength testing. For burst strength testing, grafts had thin latex water balloons inserted through the center and tied down to the burst device with 2-0 silk suture. Air was permitted to flow into the graft at a rate of 10 mmHg/min until rupture occurred, and pressure was recorded using mmHg. The burst strength results were shown in Table 3, below. All three scaffolds showed burst strength greater than 3000 mmHg.

TABLE 3
Burst strength of mesh/rolled degraded
polymer grafts after 0 day (1 hour).

| Sample | Burst Pressure (mmHg) |
|---|---|
| Mesh/Rolled melt blown tube/Mesh- 1 | 3367 |
| Mesh/Rolled melt blown tube/Mesh- 2 | 3363 |
| Mesh/Rolled melt blown tube/Mesh- 3 | 3380 |

### Example 14

### Synthesis of a Polycaprolactone (PCL) Electrospun Sheet

Solutions of 150 mg/mL of PCL (Lakeshore Biomaterials, Mw: 125 kDa, lot no.: LP563) in 1,1,1,3,3,3-hexafluoro-2-propanol (HFP, TCI America Inc.) solvent were prepared. Solutions were left in a box (dark environment) overnight on a shaker plate to ensure that all PCL had dissolved and formed a homogenous solution. 4 mL of polymer solution was then drawn into a plastic Beckton Dickinson syringe (5 ml) and placed in a KD Scientific syringe pump (Model 100) to be dispensed at a rate of 5.5 ml/hr. A high voltage power supply (Spellman CZE1000R; Spellman High Voltage Electronics Corporation) was used to apply a voltage of +22 kV to a blunt tip 18 gauge needle fixed to the solution containing syringe. Solutions were electrospun onto a 2.5 cm diameter cylindrical grounded mandrel placed 20 cm from the needle tip and rotating at a rate of ˜400 rpm to produce a scaffold of randomly oriented fibers.

Immediately after electrospinning, the mandrel and the scaffold were quickly dunked in an ethanol bath, and the scaffold was carefully slid off the mandrel. The tube (inner diameter 2.5 cm, thickness: ˜60 to 100 microns, length: 10 cm) was then placed in a fume hood for 30 minutes to allow for the evaporation of any residual ethanol. The tube was cut to make a 10 cm×10 cm sheet.

### Example 15

### Synthesis of a Scaffold of a Braided Mesh/Rolled Melt Blown 9/91 Cap/PDO Sheet/Electrostatic Spun PCL Sheet/Braided Mesh Scaffold

For the present invention, two sizes (2 mm, 3 mm) of PDO mesh tubes were fabricated at Secant Medical (Perkasie, Pa.) to form the inner and outer braided mesh tubes. Hundred micron PDO monofilament was wound onto 24 individual braiding spools and setup on one of Secant Medical's braiding machines. The 24 ends of 100 micron PDO monofilament was braided onto a 2 mm or a 3 mm mandrel having 18″ in length in a 1×1 pattern at approximately a 90° braid angle. The mandrel was then put on a rack and heat-set in an inert atmosphere oven at 85 C.° for 15 mins.

As described in Example 9, Human Umbilical Tissue cells (cell density of 1.75×106/cm2/scaffold) were seeded onto 9/91 Cap/PDO melt blown nonwoven scaffolds (3×3 cm2) (prepared as described in Example 4) and poly(caprolactone) (PCL) electrospun scaffolds (2.5×3 cm2) (prepared as described in Example 14). Cell seeded scaffolds were cultured with low glucose DMEM (Gibco), 15 percent fetal bovine serum (HyClone), GlutaMax (Gibco) and 1 percent Pen Strep (Gibco). Culture medium was changed every 2-3 days, and samples were maintained in culture dishes for up to 1 week.

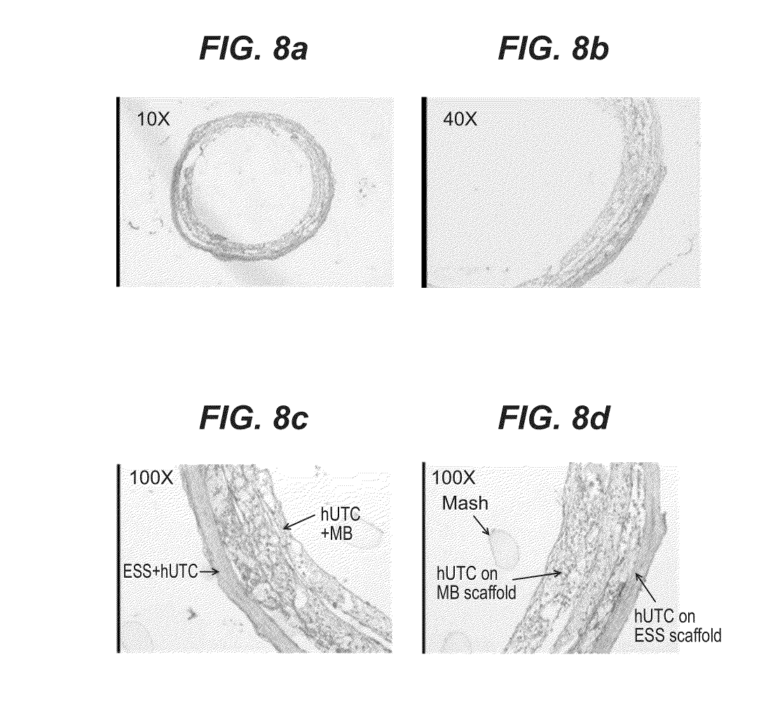

After one week of static culturing, the cell seeded melt blown nonwoven scaffold sheet was rolled onto a braided mesh (2 mm inner diameter, 24 ends of 100 micron polydioxanone monofilament, Secant Medical (Perkasie, Pa.), which was placed onto a mandrel (2 mm Teflon coated rod). On top of the rolled melt blown scaffold, the cell seeded electrospun (PCL) sheet was rolled onto the melt blown scaffold. A second braided mesh (3 mm inner diameter, 24 ends of 100 micron polydioxanone monofilament, Secant Medical) was placed onto the rolled melt blown/electrospun tubular scaffold. The scaffold was placed into bioreactor cassette and cultured for an additional week. At the end of culturing, the cell seeded scaffolds were fixed in 10 percent formalin and a cross section was stained with H&E. Histology results showed cellular infiltration within the tubular scaffold in FIGS. 8a-d.

### Example 16

### Synthesis of Segmented Epsilon-Caprolactone-Rich Poly(Epsilon-Caprolactone-co-p-Dioxanone) Triblock Copolymer at 82/18 by Mole (Cap-Rich Cap/PDO Copolymer) [Initial Feed Charge of 70/30 Cap/PDO]

The synthesis of this copolymer was done by following the procedure of Example 3 with the first stage charge of 18,078 grams of epsilon-caprolactone and 20.1 grams of diethylene glycol and 27.4 mL of a 0.33M solution of stannous octoate in toluene. In the second, copolymerization stage, 6,923 grams of molten p-dioxanone monomer was added from a melt tank.

At the end of copolymerization step, the copolymer was discharged into Teflon coated discharge containers and placed into nitrogen curing oven set at 80° C. for 22 hours for the additional solid state polymerization step. After the completion of the reaction, the trays were removed from oven, place in freezer until ready for grinding and drying steps as described in Example 3.

The dried resin exhibited an inherent viscosity of 1.74 dL/g, as measured in hexafluoroisopropanol at 25° C. and at a concentration of 0.10 g/dL. Gel permeation chromatography analysis showed a weight average molecular weight of approximately 59,400 Daltons. Nuclear magnetic resonance analysis confirmed that the copolymer resin contained 17.5 mole percent poly(p-dioxanone) and 81.9 mole percent poly(epsilon-caprolactone) with a residual monomer content of less than 1.0 percent.

### Example 17

### Monofilament Extrusion of the Segmented Epsilon-Caprolactone-Rich Poly(Epsilon Caprolactone-co-p-Dioxanone)Triblock Copolymer of Example 3 (91/9 by Mole)

The copolymer of Example 3 was extruded using a single-screw 1-inch extruder with an L/D of 18/1. The die had a diameter of 50 mils and an L/D of 5/1; the die temperature was 91° C. After an air gap of ¼ inch, the extrudate was quenched in a 20° C. water bath.

The fiber line was directed towards a first set of unheated godet rolls at a linear speed of 20 fpm. The fiber line was then directed towards a second unheated godet rolls operating at 105 fpm. The fiber line was then directed through a 6-foot hot air oven at 75° C. to a third set of unheated godet rolls; this set of rolls was operating at 160 fpm. The line was then directed through a second 6-foot hot air oven at 75° C. to a fourth set of unheated godet rolls. This last set of rolls was operating at 132 fpm, which is a lower speed than the previous set of godet rollers allowing the fiber to relax slightly (17.5%). The overall draw ratio was 6.6.

A second fiber was produced using the exact same conditions except that the two 6-foot hot air ovens were both set to a temperature 65° C.

Both resulting monofilaments appeared to be very smooth, and pliable yet strong. Tensile properties were determined using an Instron testing machine on the unannealed and annealed monofilaments of the first process (Fiber-IA and Fiber-IAa) and the unannealed monofilaments of the second process (Fiber-IB). The gage length 5 inches; a sampling rate of 20 pts/secs with a crosshead speed of 12 in/min was employed. The full scale load range was 100 lbf. Selected tensile properties (mean values) are listed In Table 4. Knot tensile measurements were made with a single knot made in the middle of the thread.

TABLE 4
Tensile Properties of Unannealed and Annealed 2-0 Monofilaments made from
the 91/9 Cap/PDO Copolymer Described in Example 3.

| | | | Final | Straight | | Knot | Young's |
|---|---|---|---|---|---|---|---|
| | Diameter | Annealing | Draw | Tensile | Elongation | Tensile | Modulus |
| Fiber | (Mils) | Conditions | Ratio | (Lbs) | (%) | (Lbs) | (Kpsi) |
| IA | 14.30 | None | 6.6x | 8.40 | 34.9 | 6.20 | 76.9 |
| IA-a | 14.17 | 55° C./6 hrs | 6.6x | 8.70 | 31.0 | 6.26 | 131.5 |
| IB | 14.34 | None | 6.6x | 8.72 | 30.2 | 6.32 | 87.3 |

### Example 18

### Monofilament Extrusion of Segmented Epsilon-Caprolactone-Rich Poly(Epsilon-Caprolactone-co-p-Dioxanone)Triblock Copolymer at 82/18 by Mole

The copolymer of Example 16 was extruded using a single-screw 1-inch extruder with an L/D of 18/1. The die had a diameter of 50 mils and an L/D of 5/1; the die temperature was 91° C. After an air gap of ¼ inch, the extrudate was quenched in a 20° C. water bath.

The copolymer of Example 16 was extruded under two different conditions using a single-screw 1-inch extruder with an L/D of 18/1; the runs differed in extrusion temperature. In both runs, the die had a diameter of 50 mils and an L/D of 50/1. In the first run, the die temperature was 140° C., and in the second, it was 150° C. After an air gap of ¼ inch, the extrudates were quenched in a 20° C. water bath. The fiber lines were directed towards a first set of unheated godet rolls at a linear speed of 20 fpm. The fiber lines were then directed towards a second unheated godet rolls operating at 106 fpm. The fiber lines were then directed through a 6-foot hot air oven at 75° C. to a third set of unheated godet rolls; this set of rolls was operating at 160 fpm. The lines were then directed through a second 6-foot hot air oven at 75° C. to a fourth set of unheated godet rolls. This last set of rolls was operating at 130 fpm, which is a lower speed than the previous set of godet rollers allowing the fiber to relax slightly (18.8%). The overall draw ratio was 6.5.

Both resulting monofilaments appeared to be very smooth, and pliable yet strong. Tensile properties were determined using an Instron testing machine on the unannealed and annealed monofilaments of the first process (Fiber-IIA and Fiber-IIAa) and the unannealed monofilaments of the second process (Fiber-IIB). The gage length 5 inches; a sampling rate of 20 pts/secs with a crosshead speed of 12 in/min was employed. The full scale load range was 100 lbf. Selected tensile properties (mean values) are listed In Table 5. Knot tensile measurements were made with a single knot made in the middle of the thread.

TABLE 5
Tensile properties of Unannealed and Annealed 2-0 Monofilaments made from
82/18 Cap/PDO copolymer described in Example 16.

| | | | Final | Straight | | Knot | Young's |
|---|---|---|---|---|---|---|---|
| | Diameter | Annealing | Draw | Tensile | Elongation | Tensile | Modulus |
| Fiber | (Mils) | Conditions | Ratio | (Lbs) | (%) | (Lbs) | (Kpsi) |
| IIA | 14.23 | None | 6.5x | 8.73 | 35.8 | 6.71 | 92.1 |
| IIA-a | 14.14 | 55° C./6 hrs | 6.5x | 8.42 | 36.9 | 6.07 | 149.7 |
| IIB | 14.16 | None | 6.5x | 8.21 | 35.6 | 6.22 | 97.4 |

The novel bioabsorbable copolymers of the present invention and novel medical devices made from such copolymers have numerous advantages. The advantages include the following: pliability of the fibers; extended breaking strength retention profile; can be made into a monofilament; low tissue reaction; easier to pull through tissue; lesser tissue drag; believed to have better moldability; dimensional stability; expected improved photostability vs. poly(p-dioxanone) homopolymer. The copolymers are readily made into long-term absorbable sutures having superior properties, both monofilament and braided constructions.

Although this invention has been shown and described with respect to detailed embodiments thereof, it will be understood by those skilled in the art that various changes in form and detail thereof may be made without departing from the spirit and scope of the claimed invention.

We claim:

1. An absorbable copolymer, comprising repeating units of polymerized p-dioxanone and polymerized epsilon-caprolactone, wherein the polymerized epsilon-caprolactone is present at a concentration of about 50 mole percent or more, and wherein said absorbable copolymer is segmented and semicrystalline.

2. The copolymer of claim 1, wherein the copolymer comprises about 5 mole percent to about 40 mole percent of p-dioxanone.

3. The copolymer of claim 1, wherein the copolymer has an inherent viscosity between about 0.5 dL/g and about 2.5 dL/g.

4. The copolymer of claim 1, wherein the copolymer has a glass transition temperature below about 25° C.

5. The copolymer of claim 1, wherein the copolymer has an absorption time between about 6 and about 24 months.

6. A medical device comprising an absorbable copolymer, said copolymer comprising repeating units of polymerized p-dioxanone and polymerized epsilon-caprolactone, wherein the polymerized epsilon-caprolactone is present at a concentration of about 50 mole percent or more, and wherein said absorbable copolymer is segmented and semicrystalline.

7. The medical device of claim 6 further comprising an active ingredient.

8. The medical device of claim 7 in which the active ingredient is an antimicrobial.

9. The medical device of claim 8 in which the antimicrobial is Triclosan.

10. A surgical suture comprising an absorbable copolymer, the copolymer comprising repeating units of polymerized p-dioxanone and polymerized epsilon-caprolactone, wherein the polymerized epsilon-caprolactone is present at a concentration of about 50 mole percent or more, and wherein said absorbable copolymer is segmented and semicrystalline.

11. The surgical suture of claim 10 further comprising an active ingredient.

12. The surgical suture of claim 11 in which the active ingredient is an antimicrobial.

13. The surgical suture of claim 12 in which the antimicrobial is Triclosan.

14. A surgical mesh comprising an absorbable copolymer, the copolymer comprising repeating units of polymerized p-dioxanone and polymerized epsilon-caprolactone, wherein the polymerized epsilon-caprolactone is present at a concentration of about 50 mole percent or more, and wherein said absorbable copolymer is segmented and semicrystalline.

15. The surgical mesh of claim 14 further comprising an active ingredient.

16. The surgical mesh of claim 15 in which the active ingredient is an antimicrobial.

17. The surgical mesh of claim 16 in which the antimicrobial is Triclosan.

18. The copolymer of claim 1 having a crystallinity level between about 10 and about 50 percent.

19. The medical device of claim 6 having a crystallinity level between about 10 and about 50 percent.

20. The surgical suture of claim 10 having a crystallinity level between about 10 and about 50 percent.

21. The surgical mesh of claim 14 having a crystallinity level between about 10 and about 50 percent.

22. The surgical suture of claim 10 wherein said suture comprises a monofilament or a braid.

23. The surgical suture of claim 22 wherein said suture comprises a monofilament with Young's modulus of less than about 150,000 psi.

24. The suture of claim 10, wherein the copolymer has an absorption time between about 6 and about 24 months.

25. The suture of claim 22, wherein the suture is a monofilament suture.

26. The suture of claim 10, wherein the copolymer comprises about 5 mole percent to about 40 mole percent of p-dioxanone.

27. The suture of claim 10, wherein the copolymer has an inherent viscosity between about 0.5 dL/g and about 2.5 dL/g.

28. The suture of claim 10, wherein the copolymer has a glass transition temperature below about 25° C.
